SEQUENCE LISTING (5 sequences)

SEQ ID NO: 1 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 2 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 3 - protein, 139 aa, Homo sapiens
LKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGSTFEERK

SEQ ID NO: 4 - protein, 133 aa, Artificial Sequence
  Vector sequence
MSSSHLFYLALCLLTFTSSTTAGPETLCGAELVDALQFVCGPRGFYFNKPTGYGSSIRRAPQTGIVDECCFRSCDLRRLEMYCAPLKPTKAARSIRAQRHTDMPKTQKSPSLSTNKKTKLQRRRKGSTFEEHK

SEQ ID NO: 5 - DNA, 890 bp, Homo sapiens
tcactgtcactgctaaattcagagcagattagagcctgcgcaatggaataaagtcctcaaaattgaaatgtgacattgctctcaacatctcccatctctctggatttccttttgcttcattattcctgctaaccaattcattttcagactttgtacttcagaagcaatgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatgtaggaagaccctcctgaggagtgaagagtgacatgccaccgcaggatcctttgctctgcacgagttacctgttaaactttggaacacctaccaaaaaataagtttgataacatttaaaagatgggcgtttcccccaatgaaatacacaagtaaacattccaacattgtctttaggagtgatttgcaccttgcaaaaatggtcctggagttggtagattgctgttgatcttttatcaataatgttctatagaaaagaaaaaaaaat

(19) United States
(12) Patent Application Publication
(10) Pub. No.: US 2010/0216709 A1
(43) Pub. Date: Aug. 26, 2010

(54) SYSTEMIC INSULIN-LIKE GROWTH FACTOR-1 THERAPY REDUCES DIABETIC PERIPHERAL NEUROPATHY AND IMPROVES RENAL FUNCTION IN DIABETIC NEPHROPATHY

(75) Inventors: Ronald K. Scheule, Framingham, MA (US); Qiuming CHU, Framingham, MA (US)

Correspondence Address: ROBINS & PASTERNAK, 1731 EMBARCADERO ROAD, SUITE 230, PALO ALTO, CA, 94303, US

(73) Assignee: GENZYME CORPORATION, Cambridge, MA (US)

(21) Appl. No.: 12/609,115

(22) Filed: Oct. 30, 2009

Related U.S. Application Data
(63) Continuation of application No. PCT/US2008/062129, filed on May 1, 2008.
(60) Provisional application No. 60/927,244, filed on May 1, 2007.
(60) Provisional application No. 60/989,213, filed on Nov. 20, 2007.

(51) Int. Cl. A61K 38/30; A61P 25/02; A61K 31/7088

(52) U.S. Cl. 514/12; 514/44 R

(57) ABSTRACT

The present invention provides methods of treatment of patients suffering from the complications of blood sugar disorders: diabetic peripheral neuropathy and diabetic nephropathy by administration of IGF-1 via protein therapy or gene therapy. It relates to methods of treating an individual having a diabetic disorder or a hyperglycemic disorder, comprising administering to the individual an effective amount of a DNA vector expressing IGF-1Eb or IGF-1Ec in vivo or an effective amount of at the IGF-1Eb or IGF-1Ec protein in the early hyperalgesia stage or in patients that have advanced to the hyposensitivity stage. Treatment at the early hyperalgesia stage prevents subsequent hyposensitivity with increases or maintenance of sensory nerve function. IGF-1Eb or IGF-1Ec treatment also increases muscle mass and improves overall mobility, which indicates a treatment-related improvement in motor function. Treatment with IGF-1Eb or IGF-1Ec at the hyposensitivity stage reverses hyposensitivity and improves muscle mass and overall health. Systemic IGF-1 provides a therapeutic modality for treating hyposensitivity associated with DPN. In addition, IGF-1Eb or IGF-1Ec provides a therapeutic modality for treating diabetic nephropathy. IGF-1Eb or IGF-1Ec improves renal function as evidenced by a modulation in serum albumin concentration and a reduction in urine volume and protein levels. IGF-1Eb or IGF-1Ec also reduces diabetic glomerulosclerosis.

### BACKGROUND

In general terms, the most common types of diabetes mellitus are Type I, Impaired Glucose Tolerance (“IGT”) and Type II. In Type I diabetes, the beta cells in the pancreas, often through an auto-immune reaction, cease producing insulin into the bloodstream of the person. Insulin is a chemical substance which is normally secreted into the bloodstream by beta cells within the pancreas. Insulin is vitally important to the person because it enables the person to properly utilize and consume sugar in the bloodstream as part of the metabolism process.

Two major forms of diabetes mellitus are now recognized. Type I diabetes, or insulin-dependent diabetes, is the result of an absolute deficiency of insulin, the hormone which regulates glucose utilization. Type II diabetes, or non-insulin-independent diabetes, often occurs in the face of normal, or even elevated levels of insulin and appears to be the result of the inability of tissues to respond appropriately to insulin. Most of the Type II diabetics are also obese. In Type I cases, where the pancreas has ceased producing insulin, it is necessary for the afflicted person to inject insulin at prescribed periodic intervals and dosages in order to control the level of sugar in the blood. Oral ingestion of insulin is also possible but usually less effective due to the degradation of insulin caused by the passage through the stomach and upper intestine.

In IGT and Type II diabetes, the pancreas continues to produce insulin but, some or all of the insulin may fail to bind to the body cell receptors and/or internalization of insulin in the cells is reduced. In such cases, there may be a sufficient level of insulin in the blood, but the ability of the cells to uptake glucose is reduced or non-existent because of reduced internalized insulin. When a Type II diabetic cell binds insulin but does not take up glucose, it indicates a defect in the signaling pathway. This results in an increased need for insulin; however, this need for insulin is not met because the β cells in a Type II diabetic are defective in that they do not secrete enough insulin.

The existence of Type I, IGT or Type II diabetes in a person is usually determined by an oral glucose tolerance test (OGTT). OGTT is a test in which the fasting patient is given a known amount of glucose (sugar) by mouth, and the blood is tested at intervals thereafter to note the quantity of sugar in the blood. A curve is then constructed from which important information about the person can be drawn. The glucose tolerance test curve will typically show whether the patient is hyperglycaemic (diabetic) or whether the patient has too little sugar in his or her blood and is therefore hypoglycaemic.

Symptoms of hyperglycaemia can be headaches, increased urination, thirst, nausea, weight loss, fatigue and coma. Hyperglycaemia can be caused by Hypoinsulinism, a condition in which the insulin producing beta cells of the pancreas fail to manufacture insulin or manufacture arid secrete a reduced amount of insulin into the bloodstream. In such cases, levels of sugar in the blood are dramatically increased.

Hyperglycaemia can also be caused by failure of some or all of the available insulin in the blood to bind to the body's cell receptors and/or internalization of insulin in the cells is reduced. Hypoglycaemia (too little sugar) is also a blood condition that diabetics must constantly guard against. The symptoms of hypoglycaemia are abrupt episodes of intense hunger, trembling of the hands and body, faintness, black spots before the eyes, mental confusion, sweating, abnormal behavior, and, in severe cases, convulsions with loss of consciousness. In such cases, examination of the blood at the time of these attacks will show an extremely low level of circulating sugar in the blood.

Insulin dependent diabetes mellitus (IDDM) is an organ specific autoimmune disease affecting close to a million people in different age groups in the United States. The disease is characterized by extensive destruction of the insulin producing beta cells in the pancreatic islets and dysregulation of glucose metabolism leading to frank diabetes. The defining feature of IDDM is the lymphocytic infiltration of the islets. Among the invading cells, T cells appear to be one of the major mediators of autoimmune destruction. Type I diabetes is further characterized by increased levels of antibodies to various islet associated antigens, including insulin, GAD65, GAD67 and ICA5 12. These antibodies can be detected much before frank disease, and an immune response to such antigens can be used as a predictor for impending diabetes in patients with susceptible genetic (HLA) haplotypes. Currently, patients are dependent on insulin injections to maintain normoglycemia.

Insulin is a polypeptide hormone consisting of two disulfide-linked chains, an A chain consisting of 21 amino acid residues and a B chain of 30 residues. While administration of insulin can provide significant benefits to patients suffering from diabetes, the short serum half-life of insulin creates difficulties for maintaining proper dosage. The use of insulin also can result in a variety of hypoglycemic side-effects and the generation of neutralizing antibodies. Lee et al., Nature 408:483-488 (2000) have created a single-chain insulin analog (SIA), which does not need to be processed, and thus is relatively simple to make recombinantly. Others, such as Thule et al. Gene Therapy 7:1744-1752 (2000) have engineered an insulin chain that is processed by furin, a ubiquitously expressed endoprotease.

Type II diabetes is a progressive, multifactorial disease which results from insulin resistance and is characterized initially by elevated fasting blood glucose levels. It is believed that genetic factors contribute to susceptibility to type II diabetes, but other important risk factors such as, obesity, aging, diet, and lack of exercise also play a role. A large number of drugs have been developed to treat hyperglycemia, including those that promote release of insulin from the pancreas, uptake of glucose from the blood, and reduction in the level of glucose production. Unfortunately, these treatments generally only slow the progression of type II diabetes, which can progress to an insulin dependent state and the development of complications associated with diabetes such as hypertension, problematic ulcerative lesions on limbs, end-stage renal failure, retinopathy and cardiovascular disease. More than 10 million people in the US alone suffer from type II diabetes, with the incidence increasing dramatically.

Diabetic peripheral neuropathy (DPN) is a particularly debilitating complication of diabetes resulting from sensory and motor neuron damage. Up to half of diabetic patients have some degree of DPN. Symptoms are typically dominated by sensory defects. Early in the disease process, DPN is manifested by hyperalgesia, but over time patients suffer from become hyposensitive, patients suffer from hypoalgesia and muscle weakness. This hyposensitivity can lead to significant morbidity by predisposing the lower extremities to injury, ulceration and eventual amputation. There are treatment options for pain relief for the early hyperalgesia stage of DPN. However, currently, there is no treatment for this later hypoalgesia, or hyposensitivity, stage.

Approximately 12-50% of diabetic patients have some degree of DPN. Approximately 15% of diabetic patients in the United States develop at least one foot ulcer during their lifetime; this number increases to approximately 25% world-wide. Of these diabetic foot ulcers, between 60-70% are primarily neuropathic in origin. Current therapies provide pain relief for early hyperalgesia. There are no interventions for late hyposensitivity. In addition, there is no effective intervention for foot ulcers thus some patients eventually require lower-limb amputation. Intensive insulin therapy to control HbA1C reduces the incidence of new clinically detected neuropathy. Despite this reduction, certain diabetic patients can still develop DPN. Intensive insulin therapy also significantly increases the risk of hypoglycemic episodes during sleep. Hypoglycemia can also contribute to the development of hyposensitivityhypoalgesia. Therapeutics to treat the hypoalgesia associated with diabetes are needed.

Diabetic nephropathy (DN) is the most common cause of end stage renal disease (ESRD) in the United State, and in 2002, accounted for over 40% of patients on dialysis. Strategies to prevent and control diabetic nephropathy would be expected to result in a reduction in patient morbidity and mortality, as well as a significant cost savings. The pathology of diabetic nephropathy manifests histologically as diabetic glomerulosclerosis, and is characterized by glomerular basement membrane thickening and mesangial expansion with increased extracellular matrix deposition (McLaughlin N G et al 2005). IGF-1 has been considered as a major contributor to the development of the disease. IGF-1 induced glomerular hypertrophy, which is early progression of the disease. IGF-1 also resulted in intracellular lipid accumulation in mesangial cells (Berfied A K et al 2002; Lupia E et al 1999).

Growth factors have been suggested as therapeutics for DPN and have been shown to promote neuronal survival, stimulate repair of peripheral nerve injury, and even induce nerve regeneration under diabetic conditions. Recombinant nerve growth factor (NGF) and brain derived neurotrophic factor have been evaluated clinically, but have not shown significant benefit. Vascular endothelial growth factor (VEGF) and C-peptide have also completed phase I clinical trials.

Insulin like growth factor 1 (IGF-1) provides trophic support for neurons of both peripheral and central nervous systems. Systemic IGF-1 levels of diabetics have been shown to be lower than those of non-diabetics, and serum IGF-1 levels in diabetics with DPN are lower than those in diabetics without DPN. Although levels of both IGF-1 and other neurotrophic factors decrease with age, IGF-1 can up-regulate many of these factors, including neurotrophin-3, platelet derived growth factor, fibroblast growth factor, IGF-2 hypoxic-inducible factor-1 alpha and VEGF. Finally, diabetics can also lose muscle mass, which contributes to deficits in motor function, while IGF-1 is myotrophic. Given these attributes of IGF-1 it is therefore important to determine whether restoring systemic IGF-1 in diabetics to more normal or higher levels provides therapeutic benefits for the neuropathy associated with diabetes.

Results in rodent models of DPN support the consideration of IGF-1 as a potential therapeutic for DPN. For example, recombinant human IGF-1 (rhIGF-1) was found to stabilize hyperalgesia in the STZ rat model of DPN. Transgenic mice deficient in IGF-1 develop DPN symptoms, and rhIGF-1 could restore both sensory and motor nerve conduction velocities in these mice. Histologically, rhIGF-1 also reversed neuroaxonal dystrophy in the STZ rat model of diabetic autonomic neuropathy. However, systemic IGF-1 has not to date been shown to provide benefit in the hypoalgesia stage of DPN, especially where ongoing hyperglycemia is a contributing factor in the disease process itself.

### SUMMARY OF THE INVENTION

The present invention provides methods of treatment of patients suffering from the complications of blood sugar disorders: diabetic peripheral neuropathy and diabetic nephropathy by administration of IGF-1 via protein therapy or gene therapy. In certain embodiments, the invention uses gene therapy vectors which provide IGF-1Eb or IGF-1Ec protein to the patient. In other embodiments, the invention provides the IGF-1Eb or IGF-1Ec protein directly to the patient. In certain embodiments of the invention, vectors are provided which comprise regulatory elements, such as promoters and enhancers that may be controlled by the levels of insulin, glucose or other biological and chemical factors in the bloodstream of the patient. The IGF-1Eb or IGF-1Ec can be delivered via DNA vectors, which may be viral or non-viral in origin. In other embodiments, the present invention relates to methods of treating an individual having a diabetic disorder or a hyperglycemic disorder, comprising administering to the individual an effective amount of a DNA vector expressing IGF-1Eb or IGF-1Ec in vivo at the early hyperalgesia stage or in patients that have advanced to the hyposensitivity stage. Treatment at the early hyperalgesia stage prevents subsequent hyposensitivity with increases or maintenance of sensory nerve function. IGF-1Eb or IGF-1Ec treatment also increases muscle mass and improves overall mobility, which indicates a treatment-related improvement in motor function. Treatment with IGF-1Eb or IGF-1Ec at the hyposensitivity stage reverses hyposensitivity and improves muscle mass and overall health. Systemic IGF-1 provides a therapeutic modality for treating hyposensitivity associated with DPN.

In addition, IGF-1Eb or IGF-1Ec provides a therapeutic modality for treating diabetic nephropathy. IGF-1Eb or IGF-1Ec improves renal function as evidenced by a modulation in serum albumin concentration and a reduction in urine volume and protein levels. IGF-1Eb or IGF-1Ec also reduces diabetic glomerulosclerosis.

### BRIEF DESCRIPTION OF THE FIGURES

FIGS. 1A-1D show detection of hypersensitivity in STZ-treated mice using the von Frey test 8 days post STZ (FIG. 1A); hot plate analysis 11 days post STZ (FIG. 1B); hot plate analysis 160 days post STZ (FIG. 1C); and cold water tests at various time points (FIG. 1D) of STZ-treated and vehicle-treated mice.

FIG. 2 shows serum IGF-1 levels in STZ-induced mice injected with plasmid DNA comprising mouse-derived IGF-1Eb cDNA operably linked to two copies of a human prothrombin enhancer and a human serum albumin promotor and non-STZ-induced mice.

FIGS. 3A-3D show bioactivity of IGF-1 in vivo in IGF-1-treated and -untreated mice. FIG. 3A shows body weight; FIG. 3B shows fat mass; FIGS. 3C and 3D show lean mass.

FIG. 4 shows sensory function in mice in the late stage of DPN measured using the hot plate assay in IGF-1 treated mice and untreated mice.

FIGS. 5A-5C show overall activity in IGF-1-treated and -untreated mice. FIG. 5A shows rearing activity; FIG. 5B shows ambulatory activity; FIG. 5C shows total mouse activity.

FIGS. 6A-6D show glucose activity (FIG. 6A), and the number and size of pancreatic islets (FIGS. 6B, 6C and 6D) in IGF-1-treated and -untreated mice. Arrows point to pancreatic islets.

FIGS. 7A-7C show histologic findings in IGF-1-treated and -untreated mice.

FIGS. 8A and 8B show percent survival versus day (FIG. 8A) and serum IGF-1 levels versus day (FIG. 8B), in IGF-1-treated and -untreated mice.

FIGS. 9A and 9B show the effect of treating diabetic mice 60 days after STZ treatment with various doses of AAV-IGF-1.

FIGS. 10A and 10B show the effect of treating diabetic mice 60 days after STZ treatment with various doses of AAV-IGF-1 on body weight (FIG. 10A) and muscle mass (FIG. 10B).

FIG. 11 shows the effect of treating diabetic mice 60 days after STZ treatment with various doses of AAV-IGF-1 on total activity.

FIG. 12 shows the effect of various dose levels of AAV-IGF-1 on hyposensitivity in diabetic mice as measured by the hot plate test.

FIGS. 13A and 13B show the effect of treating diabetic mice 60 days after STZ treatment with various doses of AAV-IGF-1 on serum IGF-1 levels (FIG. 13A) and body weight (FIG. 13B).

FIGS. 14A and 14B show the effect of treating diabetic mice 60 days after STZ treatment with various doses of AAV-IGF-1 on glucose (FIG. 14A) and HbA1C (FIG. 14B).

FIG. 15 shows serum albumin as a measure of renal function in diabetic mice 60 days after STZ treatment with various doses of AAV-IGF-1.

FIGS. 16A and 16B show urine protein (FIG. 16A) and urine volume (FIG. 16B) in diabetic mice 60 days after STZ treatment with various doses of AAV-IGF-1.

FIGS. 17A-17C show renal histology in mice 60 days after STZ treatment prior to treatment with AAV-IGF-1.

FIGS. 18A-18D show renal histology in mice following AAV-IGF-1 treatment.

FIGS. 19A-19C show hypoalgesia in STZ-treated and -untreated mice as measured by a hot plate test (FIG. 19A) and mouse tail sensory nerve conduction velocity (SNCV) (FIG. 19B) and baseline serum IGF-1 levels prior to treatment (FIG. 19C).

FIG. 20 shows HbA1c levels in IGF-1 treated and untreated mice.

FIGS. 21A and 21B show the effect of IGF-1 treatment on muscle mass. FIG. 21A shows the effect on lean mass. FIG. 21B shows the effect on fat mass.

FIGS. 22A-22H show the effect of IGF-1 treatment on skeletal muscle.

FIGS. 23A and 23B show the efficacy of systemic IGF-1 treatment using various doses of AAV-IGF-1 in mice during hypoalgesia using hot plate (FIG. 23A) and sensory nerve conduction velocity (SNCV) (FIG. 23B) assays.

FIGS. 24A-24F show the appearance of nerve fibers in normal animals (FIGS. 24A and 24D) compared to the appearance of nerve fibers in diabetic mice (FIGS. 24B and 24E) and diabetic mice treated with IGF-1 after developing hypoalgesia (FIGS. 24C and 24F).

FIGS. 25A-25C show the effects of various doses of AAV-IGF-1 on rearing activity (FIG. 25A); lean mass (FIG. 25B); and fat mass (FIG. 25C) when administered during hypoalgesia.

FIG. 26 shows the effect of a dose of 3E11 of AAV-IGF-1 on the tibialis anterior (TA) muscle mass.

FIGS. 27A-27D show histologic examination of TA fiber size in mice treated with a dose of 3E11 of AAV-IGF-1.

FIGS. 28A-28C show demyelination of ventral motor nerve fibers at the lumbar and sacral levels of the spinal cord (FIGS. 28A and 28B) in diabetic mice and that this demyelination was attenuated by IGF-1 treatment during the hypoalgesia stage of the disease (FIG. 28C).

### DETAILED DESCRIPTION OF THE INVENTION

Nucleic acid encoding IGF-1Eb or IGF-1Ec of the present invention can be administered via any gene transfer vector, such as viral vectors, including adenovirus, AAV, retrovirus and lentivirus, as well as plasmid DNA with or without a suitable lipid or polymer carriers, and is administered under conditions in which the nucleic acid is expressed in vivo. Alternatively, nucleic acid encoding IGF-1Eb or IGF-1Ec of the present invention can be administered as naked DNA or in association with an amphiphilic compound, such as lipids or compounds, or with another suitable carrier. Alternatively, the nucleic acid encoding the IGF-1Eb or IGF-1Ec can be administered ex vivo to cells (e.g., hepatocytes, myoblasts, fibroblasts, endothelial cells, keratinocytes, hematopoietic cells) of the individual and then transferred into the individual wherein the IGF-1Eb or IGF-1Ec is expressed and biologically active IGF-1Eb or IGF-1Ec is generated in vivo.

The nucleic acid (e.g., cDNA or transgene) encoding IGF-1Eb or IGF-1Ec can be cloned into an expression cassette that has a regulatory element such as a promoter (constitutive or regulatable) to drive transgene expression and a polyadenylation sequence downstream of the nucleic acid. For example, regulatory elements that are 1) specific to a tissue or region of the body; 2) constitutive; 3) glucose responsive; and/or 4) inducible/regulatable can be used. Suitable promoters include the cytomegalovirus (CMV) promoter, the CMV enhancer linked to a ubiquitin promoter such as ubiquitin B (Cubi).

Muscle-specific regulatory elements include muscle-specific promoters including mammalian muscle creatine kinase (MCK) promoter, mammalian desmin promoter, mammalian troponin I (TNNI2) promoter, or mammalian skeletal alpha-actin (ASKA) promoter. Muscle-specific enhancers useful in the present invention are selected from the group consisting of mammalian MCK enhancer, mammalian DES enhancer, and vertebrate troponin I IRE (TNI IRE, herein after referred to as FIRE) enhancer. One or more of these muscle-specific enhancer elements may be used in combination with a muscle-specific promoter of the invention to provide a tissue-specific regulatory element.

Liver-specific regulatory elements may comprise strong constitutive promoters and one or more liver-specific enhancer elements. The strong constitutive promoter may be selected from the group comprising a CMV promoter, a truncated CMV promoter, a human serum albumin promoter, and an alpha-1-anti trypsin promoter. The liver-specific enhancer elements are selected from the group comprising human serum albumin [HSA] enhancers, human prothrombin [HPrT] enhancers, alpha-1 microglobulin [A1MB] enhancers, and intronic aldolase enhancers. One or more of these liver-specific enhancer elements may be used in combination with the promoter. In one embodiment, one or more HSA enhancers are used in combination with a promoter selected from the group of a CMV promoter or an HSA promoter. In another embodiment, one or more enhancer elements selected from the group consisting of human prothrombin [HPrT] and alpha-1 microglobulin [A1MB] are used in combination with the CMV promoter. In another embodiment, the enhancer elements are selected from the group consisting of human prothrombin [HPrT] and alpha-1 microglobulin [A1MB] and are used in combination with the alpha-1-anti trypsin promoter.

Conditional promoters such as the dimerizer gene control system, based on the immunosuppressive agents FK506 and rapamycin, the ecdysone gene control system and the tetracycline gene control system. Also useful in the present invention are regulatory sequences which can regulate transcription of the IGF-1Eb or IGF-1Ec of the present invention, such as the GeneSwitch™ technology (Valentis, Inc., Woodlands, Tex.) described in Abruzzese et al., Hum. Gene Ther. 1999 10:1499-507, the disclosure of which is hereby incorporated herein by reference. With inducible or regulatable promoters, the clinician may exert additional optimization of the methods of the present invention, such that optimal levels of biologically active IGF-1Eb or IGF-1Ec are achieved.

Particular promoters are of human or mammalian origin. The promoter sequence may be a constitutive promoter, or maybe an inducible promoter. In preferred embodiments the promoter may be inducible. Particularly preferred promoter sequences for use in the present invention include liver type pyruvate kinase promoters, particularly those fragments which run (−183 to +12) or (−96 to +12) (Thompson, et al. J Biol Chem, (1991). 266:8679-82.; Cuif, et al., Mol Cell Biol, (1992). 12:4852-61); the spot 14 promoter (S14, —290 to +18) (Jump, et al., J. Biol Chem, (1990). 265:3474-8); acetyl-CoA carboxylase (O' Callaghan, et al. J. Biol Chem, (2001). 276:16033-9); fatty acid synthase (−600 to +65) (Rufo, et al., J Biol Chem, (2001). 28:28); and glucose-6-phosphatase (rat and human) (Schmoll, et al., FEBS Left, (1996). 383:63-6; Argaud, et al., Diabetes, (1996). 45:1563-71).

In particular embodiments of the present invention, the IGF-1Eb or IGF-1Ec coding sequence is further under the control of one or more enhancer elements. Among those enhancer elements which will be useful in the present invention are those which are glucose responsive, insulin responsive and/or liver specific. Particular embodiments may include the CMV enhancer; one or more glucose responsive elements, including the glucose responsive element (G1RE) of the liver pyruvate kinase (L-PK) promoter (−172 to −142); and modified versions with enhanced responsiveness (Cuif et al., supra; Lou, et al., J. Biol Chem, (1999). 274:28385-94); G1RE of L-PK with auxiliary L3 box (−172 to −126) (Diaz Guerra, et al., Mol Cell Biol, (1993). 13:7725-33; modified versions of G1RE with enhanced responsiveness with the auxiliary L3 box; carbohydrate responsive element (ChoRE) of S14 (−1448 to −1422), and modifications activated at lower glucose concentrations (Shih and Towle, J Biol Chem, (1994). 269:9380-7; Shih, et al., J Biol Chem, (1995). 270:21991-7; and Kaytor, et al., J Biol Chem, (1997). 272:7525-31; ChoRE with adjacent accessory factor site of S14 (−1467 to −1422) [et al., supra]; aldolase (+1916 to +2329)(Gregori et al., J Biol Chem, (1998). 273:25237-43; Sabourin, et al., J. Biol Chem, (1996). 271:3469-73; and fatty acid synthase (−7382 to −6970) (Rufo, et al., supra.). Preferred embodiments may also include insulin responsive elements such as glucose-6-phosphatase insulin responsive element (−780 to −722) [Ayala et al., Diabetes, (1999). 48:1885-9; and liver specific enhancer elements, such as prothrombin (940 to −860) [Chow et al., J Biol Chem, (1991) 266: 18927-33; and alpha-1-microglobulin (−2945 to −2539) [Rouet et al., Biochem J, (1998). 334:577-84).

The expression cassette is then inserted into a vector such as adenovirus, partially-deleted adenovirus, fully-deleted adenovirus, adeno-associated virus (AAV), retrovirus, lentivirus, naked plasmid, plasmid/liposome complex, etc. for delivery to the host via intravenous, intramuscular, intraportal or other route of administration. Expression vectors which can be used in the methods and compositions of the present invention include, for example, viral vectors. One of the most frequently used methods of administration of gene therapy, both in vivo and ex vivo, is the use of viral vectors for delivery of the gene. Many species of virus are known, and many have been studied for gene therapy purposes. The most commonly used viral vectors include those derived from adenoviruses, adeno associated viruses (AAV) and retroviruses, including lentiviruses, such as human immunodeficiency virus (HIV).

Adenoviral vectors for use to deliver transgenes to cells for applications such as in vivo gene therapy and in vitro study and/or production of the products of transgenes, commonly are derived from adenoviruses by deletion of the early region 1 (E1) genes (Berkner, K. L., Curr. Top. Micro. Immunol. 158 L39-66 1992). Deletion of E1 genes renders such adenoviral vectors replication defective and significantly reduces expression of the remaining viral genes present within the vector. However, it is believed that the presence of the remaining viral genes in adenoviral vectors can be deleterious to the transfected cell for one or more of the following reasons: (1) stimulation of a cellular immune response directed against expressed viral proteins, (2) cytotoxicity of expressed viral proteins, and (3) replication of the vector genome leading to cell death.

One solution to this problem has been the creation of adenoviral vectors with deletions of various adenoviral gene sequences. In particular, pseudoadenoviral vectors (PAVs), also known as ‘gutless adenovirus’ or mini-adenoviral vectors, are adenoviral vectors derived from the genome of an adenovirus that contain minimal cis-acting nucleotide sequences required for the replication and packaging of the vector genome and which can contain one or more transgenes (See, U.S. Pat. No. 5,882,877 which covers pseudoadenoviral vectors (PAV) and methods for producing PAV, incorporated herein by reference). Such PAVs, which can accommodate up to about 36 kb of foreign nucleic acid, are advantageous because the carrying capacity of the vector is optimized, while the potential for host immune responses to the vector or the generation of replication-competent viruses is reduced. PAV vectors contain the 5′ inverted terminal repeat (ITR) and the 3′ ITR nucleotide sequences that contain the origin of replication, and the cis-acting nucleotide sequence required for packaging of the PAV genome, and can accommodate one or more transgenes with appropriate regulatory elements, e.g. promoter, enhancers, etc.

Other, partially deleted adenoviral vectors provide a partially-deleted 5 adenoviral (termed “DeAd”) vector in which the majority of adenoviral early genes required for virus replication are deleted from the vector and placed within a producer cell chromosome under the control of a conditional promoter. The deletable adenoviral genes that are placed in the producer cell may include E1A/E1B, E2, E4 (only ORF6 and ORF6/7 need be placed into the cell), pIX and pIVa2. E3 may also be deleted from the vector, but since it is not required for vector production, it can be omitted from the producer cell. The adenoviral late genes, normally under the control of the major late promoter (MLP), are present in the vector, but the MLP may be replaced by a conditional promoter.

Conditional promoters suitable for use in DeAd vectors and producer cell lines include those with the following characteristics: low basal expression in the uninduced state, such that cytotoxic or cytostatic adenovirus genes are not expressed at levels harmful to the cell; and high level expression in the induced state, such that sufficient amounts of viral proteins are produced to support vector replication and assembly. Preferred conditional promoters suitable for use in DeAd vectors and producer cell lines include the dimerizer gene control system, based on the immunosuppressive agents FK506 and rapamycin, the ecdysone gene control system and the tetracycline gene control system. Also useful in the present invention may be the GeneSwitch™ technology [Valentis Inc., Woodlands, Tex.] described in Abruzzese et al., Hum. Gene Ther. 1999 10:1499-507, the disclosure of which is hereby incorporated herein by reference.

The partially deleted adenoviral expression system is further described in W099/57296.

Adenoviral vectors, such as PAVs and DeAd vectors, have been designed to take advantage of the desirable features of adenovirus which render it a suitable vehicle for delivery of nucleic acids to recipient cells. Adenovirus is a non-enveloped, nuclear DNA virus with a genome of about 36 kb, which has been well-characterized through studies in classical genetics and molecular biology (Hurwitz, M. S., Adenoviruses Virology, 3^{rd} edition, Fields et al., eds., Raven Press, New York, 1996; Hitt, M. M. et al., Adenovirus Vectors, The Development of Human Gene Therapy, Friedman, T. ed., Cold Spring Harbor Laboratory Press, New York 1999). The viral genes are classified into early (designated E1-E4) and late (designated L1-L5) transcriptional units, referring to the generation of two temporal classes of viral proteins. The demarcation of these events is viral DNA replication. The human adenoviruses are divided into numerous serotypes (approximately 47, numbered accordingly and classified into 6 groups: A, B, C, D, E and F), based upon properties including hemaglutination of red blood cells, oncogenicity, DNA and protein amino acid compositions and homologies, and antigenic relationships.

Recombinant adenoviral vectors have several advantages for use as gene delivery vehicles, including tropism for both dividing and non-dividing cells, minimal pathogenic potential, ability to replicate to high titer for preparation of vector stocks, and the potential to carry large inserts (Berkner, K. L., Curr. Top. Micro. Immunol. 158:39-66, 1992; Jolly, D., Cancer Gene Therapy 1:51-64 1994).

PAVs have been designed to take advantage of the desirable features of adenovirus which render it a suitable vehicle for gene delivery. While adenoviral vectors can generally carry inserts of up to 8 kb in size by the deletion of regions which are dispensable for viral growth, maximal carrying capacity can be achieved with the use of adenoviral vectors containing deletions of most viral coding sequences, including PAVs. See U.S. Pat. No. 5,882,877 of Gregory et al.; Kochanek et al., Proc. Natl. Acad. Sci. USA 93:5731-5736, 1996; Parks et al., Proc. Natl. Acad. Sci. USA 93:13565-13570, 1996; Lieber et al., J. Virol. 70:8944-8960, 1996; Fisher et al., Virology 217:11-22, 1996; U.S. Pat. No. 5,670,488; PCT Publication No. W096/33280, published Oct. 24, 1996; PCT Publication No. W096/40955, published Dec. 19, 1996; PCT Publication No. W097/25446, published Jul. 19, 1997; PCT Publication No. W095/29993, published Nov. 9, 1995; PCT Publication No. W097/00326, published Jan. 3, 1997; Morral et al., Hum. Gene Ther. 10:2709-2716, 1998.

Since PAVs are deleted for most of the adenovirus genome, production of PAVs requires the furnishing of adenovirus proteins in trails which facilitate the replication and packaging of a PAV genome into viral vector particles. Most commonly, such proteins are provided by infecting a producer cell with a helper adenovirus containing the genes encoding such proteins.

However, such helper viruses are potential sources of contamination of a PAV stock during purification and can pose potential problems when administering the PAV to an individual if the contaminating helper adenovirus can replicate and be packaged into viral particles.

The use of adenoviruses for gene therapy is described, for example, in U.S. Pat. No. 5,882,877; the disclosures of which are hereby incorporated herein by reference.

Adeno-associated virus (AAV) is a single-stranded human DNA parvovirus whose genome has a size of 4.6 kb. Recombinant AAV vectors are derived from single-stranded (ss) DNA parvoviruses that are nonpathogenic for mammals (reviewed in Muzyscka (1992) Curr. Top. Microb. Immunol., 158:97-129). The AAV genome contains two major genes: the rep gene, which codes for the rep proteins (Rep 76, Rep 68, Rep 52, and Rep 40) and the cap gene, which codes for AAV replication, rescue, transcription and integration, while the cap proteins form the AAV viral particle. AAV derives its name from its dependence on an adenovirus or other helper virus (e.g., herpesvirus) to supply essential gene products that allow AAV to undergo a productive infection, i.e., reproduce itself in the host cell. In the absence of helper virus, AAV integrates as a provirus into the host cell's chromosome, until it is rescued by superinfection of the host cell with a helper virus, usually adenovirus (Muzyczka, Curr. Top. Micro. Immunol. 158:97-127, 1992). It may also remain expressed episomally. Recombinant AAV-based vectors have the rep and cap viral genes that account for 96% of the viral genome removed, leaving the two flanking 145-basepair (bp) inverted terminal repeats (ITRs), which are used to initiate viral DNA replication, packaging and integration. A single AAV particle can accommodate up to 5 kb of ssDNA, therefore leaving about 4.5 kb for a transgene and regulatory elements, which is typically sufficient. However, trans-splicing systems as described, for example, in U.S. Pat. No. 6,544,785, may nearly double this limit.

Interest in AAV as a gene transfer vector results from several unique features of its biology. At both ends of the AAV genome is a nucleotide sequence known as an inverted terminal repeat (ITR), which contains the cis-acting nucleotide sequences required for virus replication, rescue, packaging and integration. There are other advantages to the use of AAV for gene transfer. The host range of AAV is broad. Moreover, unlike retroviruses, AAV can infect both quiescent and dividing cells. In addition, AAV has not been associated with human disease, obviating many of the concerns that have been raised with retrovirus derived gene transfer vectors. Alternative tissue tropisms have been demonstrated for the different AAV serotype. For example Chao et al (Mol. Ther. 2000, 2:619-23) demonstrated that AAV1 when injected into skeletal muscle can direct expression of FIX into the blood that is several logs higher than that obtained with AAV2.

In the methods of the invention, AAV of any serotype can be used. The serotype of the viral vector used in certain embodiments of the invention is selected from the group consisting from AAV1, AAV2, AAV3, AAV4, AAV5, AAV6, AAV7, and AAV8 (see, e.g., Gao et al. (2002) PNAS, 99:11854-11859; and Viral Vectors for Gene Therapy: Methods and Protocols, ed. Machida, Humana Press, 2003). Other serotype besides those listed herein can be used. Furthermore, pseudotyped AAV vectors may also be utilized in the methods described herein. Pseudotyped AAV vectors are those which contain the genome of one AAV serotype in the capsid of a second AAV serotype; for example, an AAV vector that contains the AAV2 capsid and the AAV1 genome or an AAV vector that contains the AAV5 capsid and the AAV 2 genome. (Auricchio et al., (2001) Hum. Mol. Genet., 10 (26):3075-81.)

In an illustrative embodiment, AAV is AAV2 or AAV1 or AAV8. Adeno-associated virus of many serotypes, especially AAV2, have been extensively studied and characterized as gene therapy vectors. Those skilled in the art will be familiar with the preparation of functional AAV-based gene therapy vectors. Numerous references to various methods of AAV production, purification and preparation for administration to human subjects can be found in the extensive body of published literature (see, e.g., Viral Vectors for Gene Therapy: Methods and Protocols, ed. Machida, Humana Press, 2003).

Standard approaches to the generation of recombinant rAAV vectors have required the coordination of a series of intracellular events: transfection of the host cell with an rAAV vector genome containing a transgene of interest flanked by the AAV ITR sequences, transfection of the host cell by a plasmid encoding the genes for the AAV rep and cap proteins which are required in trans, and infection of the transfected cell with a helper virus to supply the non-AAV helper functions required in trans (Muzyczka, N., Curr. Top. Micro Immunol. 158:97-129, 1992). The adenoviral (or other helper virus) proteins activate transcription of the AAV rep gene, and the rep proteins then activate transcription of the AAV cap genes. The cap proteins then utilize the ITR sequences to package the rAAV genome into an rAAV viral particle. Therefore, the efficiency of packaging is determined, in part, by the availability of adequate amounts of the structural proteins, as well as the accessibility of any cis-acting packaging sequences required in the rAAV vector genome.

Other approaches to improving the production of rAAV vectors include the use of helper virus induction of the AAV helper proteins (Clark, et al., Gene Therapy 3:1124-1132, 1996) and the generation of a cell line containing integrated copies of the rAAV vector and AAV helper genes so that infection by the helper virus initiates rAAV production. (Clark et al., Human Gene Therapy 6:1329-1341, 25 1995).

rAAV vectors have been produced using replication-defective helper adenoviruses which contain the nucleotide sequences encoding the rAAV vector genome (U.S. Pat. No. 5,856,152 issued Jan. 5, 1999) or helper adenoviruses which contain the nucleotide sequences, encoding the AAV helper proteins (PCT International Publication W095/06743, published Mar. 9, 1995) or helper herpes virus vectors (e.g. herpes simplex virus) (U.S. Pat. No. 6,686,200). Production strategies which combine high level expression of the AAV helper genes and the optimal choice of cis-acting nucleotide sequences in the rAAV vector genome have been described (PCT International Application No. W097/09441 published Mar. 13, 1997).

Other approaches include a procedure that does not include a helper virus wherein the necessary genes for AAV production are subcloned into DNA plasmids which are transfected into a cell during rAAV vector production (Salvetti et al., Hum. Gene Ther. 9:695-706, 1998; Grimm, et al., Hum. Gene Ther. 9:2745-2760, 1998; W097/09441; U.S. Pat. No. 6,632,670). The use of AAV for gene therapy is described, for example, in U.S. Pat. No. 5,753,500, the disclosures of each of the above are hereby incorporated herein by reference.

Other methods for delivery of nucleic acid to cells do not use viruses for delivery. For example, cationic amphiphilic compounds can be used to deliver the nucleic acid of the present invention. Because compounds designed to facilitate intracellular delivery of biologically active molecules must interact with both non polar and polar environments (in or on, for example, the plasma membrane, tissue fluids, compartments within the cell, and the biologically active molecular itself), such compounds are designed typically to contain both polar and non-polar domains. Compounds having both such domains may be termed amphiphiles, and many lipids and synthetic lipids that have been disclosed for use in facilitating such intracellular delivery (whether for in vitro or in vivo application) meet this definition. One particularly important class of such amphiphiles is the cationic amphiphiles. In general, cationic amphiphiles have polar groups that are capable of being positively charged at or around physiological pH, and this property is understood in the art to be important in defining how the amphiphiles interact with the many types of biologically active (therapeutic) molecules including, for example, negatively charged polynucleotides such as DNA.

Examples of cationic amphiphilic compounds that have both polar and non polar domains and that are stated to be useful in relation to intracellular delivery of biologically active molecules are found, for example, in the following references, which contain also useful discussion of (1) the properties of such compounds that are understood in the art as making them suitable for such applications, and (2) the nature of structures, as understood in the art, that are formed by complexing of such amphiphiles with therapeutic molecules intended for intracellular delivery.
(1) Feigner, et al., Proc. Natl. Acad. Sci. USA, 84, 7413-7417 (1987) disclose use of positively-charged synthetic cationic lipids including N->1 (2,3-dioleyloxy)propyl 1-N,N,N-trimethylammonium chloride (“DOTMA”), to form lipid/DNA complexes suitable for transfections. See also Feigner et al., The Journal of Biological Chemistry, 269(4), 2550-2561 (1994).
(2) Behr et al., Proc. Natl. Acad. Sci USA, 86, 6982-6986 (1989) disclose numerous amphiphiles including dioctadecylamidologlycylspermine (“DOGS”).
(3) U.S. Pat. No. 5,283,185 to Epand et al. describes additional classes and 30 species of amphiphiles including 3.betaN-(N.sup.1,N.sup.1-dimethylaminoethane)carbamoyl 1 cholesterol, termed “DC-chol”.
(4) Additional compounds that facilitate transport of biologically active molecules into cells are disclosed in U.S. Pat. No. 5,264,618 to Feigner et al. See also Feigner et al., The Journal of Biological Chemistry, 269 (4), pp. 2550-2561 (1994) for disclosure therein of further compounds including “DMRIE” 1,2-dimyristyloxypropyl-3-dimethyl-hydroxyethyl ammonium bromide.
(5) Reference to amphiphiles suitable for intracellular delivery of biologically active molecules is also found in U.S. Pat. No. 5,334,761 to Gebeyehu et al., and in Feigner et al., Methods (Methods in Enzymology), 5, 67-75 (1993). The use of compositions comprising cationic amphiphilic compounds for gene delivery is described, for example, in U.S. Pat. No. 5,049,386; U.S. Pat. No. 5,279,833; U.S. Pat. No. 5,650,096; U.S. Pat. No. 5,747,471; U.S. Pat. No. 5,767,099; U.S. Pat. No. 5,910,487; U.S. Pat. No. 5,719,131; U.S. Pat. No. 5,840,710; U.S. Pat. No. 5,783,565; U.S. Pat. No. 5,925,628; U.S. Pat. No. 5,912,239; U.S. Pat. No. 5,942,634; U.S. Pat. No. 5,948,925; U.S. Pat. No. 6,022,874; U.S. Pat. No. 5,994,317; U.S. Pat. No. 5,861,397; U.S. Pat. No. 5,952,916; U.S. Pat. No. 5,948,767; U.S. Pat. No. 5,939,401; and U.S. Pat. No. 5,935,936, the disclosures of which are hereby incorporated herein by reference.

In addition, nucleic acid encoding IGF-1Eb or IGF-1Ec of the present invention can be delivered using “naked DNA”. Methods for delivering a non-infectious, non-integrating nucleic acid sequence encoding a desired polypeptide or peptide operably linked to a promoter, free from association with transfection-facilitating proteins, viral particles, liposomal formulations, charged lipids and calcium phosphate precipitating agents are described in U.S. Pat. No. 5,580,859; U.S. Pat. No. 5,963,622; U.S. Pat. No. 5,910,488; the disclosures of which are hereby incorporated herein by reference.

Gene transfer systems that combine viral and nonviral components have also been reported. Cristiano et al., (1993) Proc. Natl. Acad. Sci. USA 90:11548; Wu et al., (1994) J. Biol. Chem. 269:11542; Wagner et al., (1992) Proc. Natl. Acad. Sci. USA 89:6099; Yoshimura et al., (1993) J. Biol. Chem. 268:2300; Curiel et al., (1991) Proc. Natl. Acad. Sci. USA 88:8850; Kupfer et al., (1994) Human Gene Ther. 5:1437; and Gottschalk et al., (1994) Gene Ther. 1:185. In most cases, adenovirus has been incorporated into the gene delivery systems to take advantage of its endosomolytic properties. The reported combinations of viral and nonviral components generally involve either covalent attachment of the adenovirus to a gene delivery complex or co-internalization of unbound adenovirus with cationic lipid:DNA complexes.

IGF-1Eb or IGF-1Ec may also be administered as a protein. The administration may be mediated by infusion. One way to deliver via infusion is with the use of a pump. Such pumps are commercially available, for example, from Alzet (Cupertino, Calif.) or Medtronic (Minneapolis, Minn.). The pump may be implantable or external. Another convenient way to administer the enzymes, is to use a cannula or a catheter. The cannula or catheter may be used for multiple administrations separated in time. Cannulae and catheters can be implanted into the body. It is contemplated that multiple administrations will be used to treat the typical patient with DPN or DN complications. Catheters and pumps can be used separately or in combination. Catheters can be inserted surgically, as is known in the art. The pump may have settings suitable for delivery rates based on the individual subject's requirements.

The present invention also provides compositions (e.g., pharmaceutical compositions) comprising the IGF-1Eb or IGF-1Ec proteins described herein. The compositions described herein can also include a pharmaceutically acceptable carrier. The terms “pharmaceutically acceptable carrier” or “carrier” refer to any generally acceptable excipient or drug delivery device that is relatively inert and non-toxic. Exemplary carriers include calcium carbonate, sucrose, dextrose, mannose, albumin, starch, cellulose, silica gel, polyethylene glycol (PEG), dried skim milk, rice flour, magnesium strearate and the like. Other suitable carriers (e.g., pharmaceutical carriers) include, but are not limited to sterile water, salt solutions (such as Ringer's solution), alcohols, gelatin, carbohydrates such as lactose, amylose or starch, talc, silicic acid, viscous paraffin, fatty acid esters, hydroxymethylcellulose, polyvinyl pyrolidone, etc. Such preparations can be sterilized and, if desired, mixed with auxiliary agents, e.g., lubricants, preservatives, stabilizers, wetting agents, emulsifiers, salts for influencing osmotic pressure, buffers, coloring and/or aromatic substances and the like which do not deteriously react with the IGF-1Eb or IGF-1Ec protein. A carrier (e.g., a pharmaceutically acceptable carrier) is preferred, but not necessary to administer the DNA vector encoding IGF-1Eb or IGF-1Ec. Suitable formulations and additional carriers are described in Remington's Pharmaceutical Sciences (17^{th} Ed., Mack Publ. Co., Easton, Pa.), the teachings of which are incorporated herein by reference in their entirety.

As described herein, an “effective amount” of DNA vectors encoding the IGF-1Eb or IGF-1Ec or an “effective amount” of the IGF-1Eb or IGF-1Ec protein is an amount such that when administered, it provides biologically active IGF-1Eb or IGF-1Ec, which interrupts the progression of diabetic peripheral neuropathy (DPN) or reverses or modulates the symptoms of DPN in the individual to whom it is administered relative to the symptoms of DPN prior to IGF-1Eb or IGF-1Ec administration. As described herein, an “effective amount” of DNA vectors encoding the IGF-1Eb or IGF-1Ec or an “effective amount” of the IGF-1Eb or IGF-1Ec protein is an amount such that when administered, it provides biologically active IGF-1Eb or IGF-1Ec, which interrupts the progression of diabetic nepropathy (DN) or reverses or modulates the symptoms of DN in the individual to whom it is administered relative to the symptoms of DN prior to IGF-1Eb or IGF-1Ec administration.

In addition, the amount of IGF-1Eb or IGF-1Ec administered to an individual will vary depending on a variety of factors, including the size, age, body weight, general health, sex and diet of the individual. In the particular embodiments wherein adenoviral or AAV vectors are used, the dose of the nucleic acid encoding IGF-1Eb or IGF-1Ec can be delivered via adenoviral or AAV particles, generally in the range of about 10⁶ to about 10¹⁵ particles, more preferably in the range of about 10⁸ to about 10¹³ particles. When nucleic acid is delivered in the form of plasmid DNA, a useful dose will generally range from about 1 ug to about 1 g of DNA, preferably in the range from about 100 ug to about 100 mg of DNA. The skilled clinician may also determine the suitable dosage based upon expression levels geared to meet particular plasma concentration levels of IGF-1Eb or IGF-1Ec. IGF-1Eb or IGF-1Ec expression can be controlled using known techniques, such as the Valentis GeneSwitch 4.0 expression vector. In addition, methods for measuring the plasma concentration levels of IGF-1Eb or IGF-1Ec are known in the art, and can be used to monitor and/or tailor the dosage regimen appropriately.

The vector encoding IGF-1Eb or IGF-1Ec can be administered using a variety of routes of administration. For example, the IGF-1Eb or IGF-1Ec can be administered intravenously, parenterally, intramuscularly, subcutaneously, orally, nasally, by inhalation, by implant, by injection and/or by suppository. The composition can be administered in a single dose or in more that one dose over a period of time to confer the desired effect. By means of the above embodiments, IGF-1Eb or IGF-1Ec is thus expressed in a cell in vivo upon introduction of the vector via intravenous, intramuscular, intraportal or other route of administration.

The present invention also provides compositions (e.g., pharmaceutical compositions) comprising the vectors encoding the IGF-1Eb or IGF-1Ec described herein. The compositions described herein can also include a pharmaceutically acceptable carrier. The terms “pharmaceutically acceptable carrier” or “carrier” refer to any generally acceptable excipient or drug delivery device that is relatively inert and non-toxic. Exemplary carriers include calcium carbonate, sucrose, dextrose, mannose, albumin, starch, cellulose, silica gel, polyethylene glycol (PEG), dried skim milk, rice flour, magnesium strearate and the like.

Other suitable carriers (e.g., pharmaceutical carriers) include, but are not limited to sterile water, salt solutions (such as Ringer's solution), alcohols, gelatin, carbohydrates such as lactose, amylose or starch, talc, silicic acid, viscous paraffin, fatty acid esters, hydroxymethylcellulose, polyvinyl pyrolidone, etc. Such preparations can be sterilized and, if desired, mixed with auxiliary agents, e.g., lubricants, preservatives, stabilizers, wetting agents, emulsifiers, salts for influencing osmotic pressure, buffers, coloring and/or aromatic substances and the like which do not deteriously react with the DNA vector encoding IGF-1Eb or IGF-1Ec. A carrier (e.g., a pharmaceutically acceptable carrier) is preferred, but not necessary to administer the DNA vector encoding IGF-1Eb or IGF-1Ec. Suitable formulations and additional carriers are described in Remington's Pharmaceutical Sciences (17^{th} Ed., Mack Publ. Co., Easton, Pa.), the teachings of which are incorporated herein by reference in their entirety.

The present invention also relates to an expression vector comprising nucleic acid encoding IGF-1Eb or IGF-1Ec wherein the vector leads to generation of IGF-1Eb or IGF-1Ec in vivo. Other embodiments include vectors, viruses and host cells comprising nucleic acids which encode a nucleic acid sequence encoding for IGF-1Eb or IGF-1Ec operably linked to one or more promoters. For example, promoters that are 1) specific to a tissue or region of the body; 2) constitutive; 3) glucose responsive; and/or 4) inducible/regulatable can be used. Suitable promoters include the cytomegalovirus (CMV) promoter, the CMV enhancer linked to the ubiquitin promoter (Cubi).

Tissue-specific regulatory elements for the muscle include muscle-specific promoters including mammalian muscle creatine kinase (MCK) promoter, mammalian desmin promoter, mammalian troponin I (TNNI2) promoter, or mammalian skeletal alpha-actin (ASKA) promoter. Muscle-specific enhancers useful in the present invention are selected from the group consisting of mammalian MCK enhancer, mammalian DES enhancer, and vertebrate troponin I IRE (TNI IRE, herein after referred to as FIRE) enhancer. One or more of these muscle-specific enhancer elements may be used in combination with a muscle-specific promoter of the invention to provide a tissue-specific regulatory element.

Liver-specific regulatory elements may comprise strong constitutive promoters and one or more liver-specific enhancer elements. The strong constitutive promoter may be selected from the group comprising a CMV promoter, a truncated CMV promoter, a human serum albumin promoter, and an alpha-1-anti trypsin promoter. The liver-specific enhancer elements are selected from the group comprising human serum albumin [HSA] enhancers, human prothrombin [HPrT] enhancers, alpha-1 microglobulin [A1MB] enhancers, and intronic aldolase enhancers. One or more of these liver-specific enhancer elements may be used in combination with the promoter. In one embodiment, one or more HSA enhancers are used in combination with a promoter selected from the group of a CMV promoter or an HSA promoter. In another embodiment, one or more enhancer elements selected from the group consisting of human prothrombin [HPrT] and alpha-1 microglobulin [A1MB] are used in combination with the CMV promoter. In another embodiment, the enhancer elements are selected from the group consisting of human prothrombin [HPrT] and alpha-1 microglobulin [A1MB] and are used in combination with the alpha-1-anti trypsin promoter, muscle specific promoters (Souza et al., Molec. Ther., 5 (5) part 2:S409 (June 2002)), liver specific promoters (WO 01/36620), and conditional promoters such as the dimerizer gene control system, based on the immunosuppressive agents FK506 and rapamycin, the ecdysone gene control system and the tetracycline gene control system. Other examples of promoters include the glucose-6-phosphatase promoter; liver type pyruvate kinase promoter; spot 14 promoter; and the acetyl-CoA carboxylase promoter. In other embodiments, the vectors, viruses and host cells of the invention may additionally be operably linked to one or more enhancers selected from the group consisting of an aldolase enhancer, glucose inducible response elements: Cho response elements; fatty acid synthase; prothrombin; alpha-1-microglobulin; and glucose-6-phosphatase. The vectors, constructs and viruses of the present invention may be assayed in hepatoma cells, such as H1141E cells.

The insulin-like growth factor (IGF-1) gene has a complex structure, which is well-known in the art. It has several alternatively spliced mRNA products arising from the gene transcript. The mature form of IGF-1 is a 70 amino acid polypeptide. The alternatively spliced isoforms generally contain the 70 amino acid mature peptide, but differ in the sequence and length of their carboxyl-terminal extensions. The peptide sequences of several known human isoforms are represented by SEQ ID NOS: 1, 2, and 3 respectively. The genomic and functional cDNAs of human IGF-1, as well as additional information regarding the IGF-1 gene and its products, are available at Unigene Accession No. NM_{—}00618. The insulin-like growth factor (IGF-1) gene has a complex structure, which is well-known in the art. It has several alternatively spliced mRNA products arising from the gene transcript. The mature form of IGF-1 is a 70 amino acid polypeptide. The alternatively spliced isoforms generally contain the 70 amino acid mature peptide, but differ in the sequence and length of their carboxyl-terminal extensions. The peptide sequences of several known human isoforms are represented by SEQ ID NOS: 1, 2, and 3 respectively. The genomic and functional cDNAs of human IGF-1, as well as additional information regarding the IGF-1 gene and its products, are available at Unigene Accession No. NM_{—}00618 an example of a gene sequence for human IGF-1 is represented by SEQ ID NO: 5. The IGF-1 protein may have the sequence shown in SEQ ID NO: 1, 2, 3, or 4 or allelic variants thereof; the cDNA in the expression cassette used in a gene therapy vector may encode for a protein sequence shown in SEQ ID NO: 1, 2, 3, or 4 or allelic variants thereof. Allelic variants may differ by a single or a small number of amino acid residues, typically less than 5, less than 4, less than 3 residues. The IGF-1 protein sequence used in the experiments is represented by SEQ ID NO: 4.

The IGF-1 protein may have the sequence shown in SEQ ID NO: 1, 2, 3, or 4 or allelic variants thereof; the cDNA in the expression cassette used in a gene therapy vector may encode for a protein sequence shown in SEQ ID NO: 1, 2, 3, or 4 or allelic variants thereof. Allelic variants may differ by a single or a small number of amino acid residues, typically less than 5, less than 4, less than 3 residues. The IGF-1 protein sequence used in the experiments is represented by SEQ ID NO: 4.

### EXAMPLES

Streptozotocin (STZ), an antibiotic produced by Streptomyces achromogenes, is one of the most widely used diabetes mellitus-inducing agents in experimental animals (described in Like, A. A., and Rossini, A. A. (1976) Streptozotocin-induced pancreatic insulitis: new model of diabetes mellitus. Science 193, 415-417.) In rodent models, streptozotocin (STZ) treatment ablates the insulin producing beta cells of the pancreas and results in a severely diabetic phenotype with DPN as a complication. The animals develop early hyperalgesia followed by hyposensitivity, analogous to patient symptoms.

DPN animal models were induced with streptozotocin (STZ) treatment. STZ treatment resulted in a severe diabetic phenotype with high blood glucose (>500 mg/dL) and a significant body weight decrease (˜6% weight loss within a week). In this STZ-induced rodent DPN model, two phases of sensory function changes were observed- hyper- and hyposensitive responses. Nerve damage mediates the observed neuropathy. Initially, the nerve damage can result in hypersensitivity (known also as hyperalgesia). Over time, this hypersensitivity may subside and is replaced with numbness (hyposensitivity). In this STZ-induced mouse model, hypersensitive responses occur approximately day 5 post-STZ (data not shown), and become more severe approximately day 14 post-STZ. After 4 to 6 weeks, the sensory response becomes hyposensitive (known also as hypoalgesia).

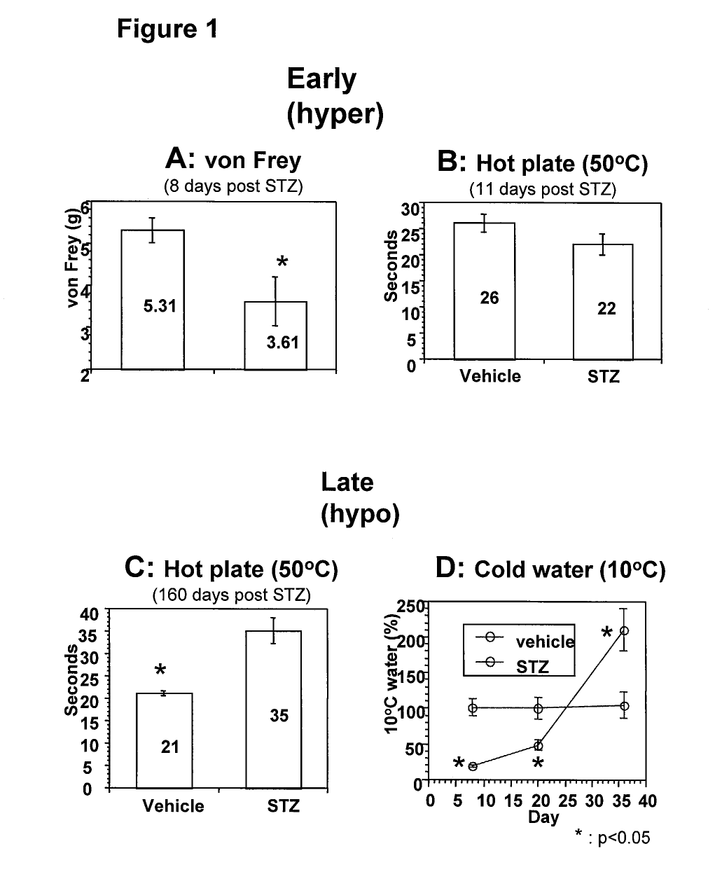

In mice treated with STZ, hyperalgesia was detected as early as 5 days post-STZ (data not shown). At day 8, STZ treated mice displayed a significantly increased sensitivity to mechanical stimulation as measured by von Frey analysis (3.61±0.58 g compared to 5.31±0.3 g for vehicle treated mice; FIG. 1A). In this diabetic condition, early hypersensitivity could be detected with von Frey (FIG. 1A) and cold water tests (FIG. 1D) but not with a hot plate analysis (FIG. 1B). The STZ-treated mice were more sensitive to thermal stimulation with cold water at day 8, viz., latency decreased to only 19±2% of vehicle treated mice (FIG. 1D). Late hyposensitivity also developed in this model, which could be detected with the hot plate (FIG. 1C) and the cold water tests (FIG. 1D). The observed responses are consistent with both literature reports and parallel clinical observations.

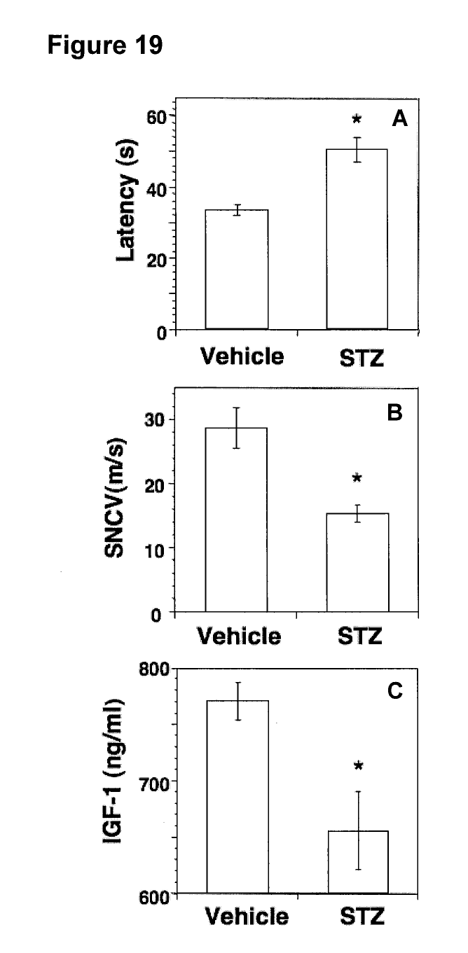

Hypoalgesia developed after another four weeks, and could be demonstrated with cold water at day 36 (FIG. 1D). At this time point, latency in STZ mice had increased to 210±30% of vehicle treated mice. Hypoalgesia was further confirmed at day 64 with another thermal stimulation assay, namely, a hot plate test (FIG. 19A). Latency in STZ mice was 50±3.5 s, significantly longer than that of vehicle treated mice (33±1.6 s). Electrophysiologic recordings also documented a significant slowing of mouse tail sensory nerve conduction velocity (SNCV) at day 115 in STZ mice (15.2±1.4 m/s), compared with vehicle treated mice (28.4±3.2 m/s; FIG. 19B). To establish baseline levels of serum IGF-1 in mice prior to treatment, serum IGF-1 levels were measured. These levels in STZ mice were significantly lower than those of vehicle treated mice, 654±35 ng/ml vs. 770±17 ng/ml, respectively, at day 28 (FIG. 19C), results consistent with the effects of STZ in rats. These data confirm that the STZ treatment paradigm used generated a severely diabetic mouse with reduced circulating IGF-1, an early hyperalgesic state followed by a later state of hypoalgesia.

Previous studies in a rat STZ model have shown that systemic administration of recombinant, mature form IGF-1 protein (70 amino acid form) stabilizes the progression of hyperalgesia. We asked whether or not one of the alternatively spliced isoforms of IGF-1 could mediate different effects. Experiments evaluated whether or not one of the alternatively spliced isoforms of IGF-1 could modulate the hyposensitivity that characterizes the later stage of DPN in STZ-treated mice. Our ultimate goal was to evaluate whether increasing systemic IGF-1 using a gene based approach in the STZ mouse model could treat the late stage disease symptoms effectively, namely hypoalgesia and muscle weakness. We first asked whether delivering a gene containing mouse IGF-1 during the hyperalgesia stage could not only interrupt disease progression but whether the systemic protein could also prevent later hypoalgesia. We then asked whether introducing a gene-based increase in systemic IGF-1 during the hypoalgesia stage could attenuate hypoalgesia and improve motor function even in the presence of ongoing hyperglycemia.

Briefly, the plasmid vector contains the hepatocyte-restricted DC190 (pDC190) expression cassette containing a human serum albumin promoter (nucleotides −486 to +20) to which are appended two copies of the human prothrombin enhancer (nucleotides −940 to −860). The cDNA for synthetic mouse IGF-1(Eb) was inserted to obtain pDC190-smIGF-1. To create the recombinant AAV vector, the IGF-1 expression cassette was cloned into the pre-viral plasmid pAAV/SP70 within the AAV2 inverted terminal repeat sequences. A fragment of the human alpha-1-antitrypsin intron was included as “staffer sequence” to make the size of the vector similar to the wild type AAV2 genome. The DC190-IGF-1 vector DNA was packaged into AAV8 capsids using a standard triple transfection protocol. The AAV8 pseudotyped vector was purified by iodixanol gradient centrifugation followed by ion exchange chromatography over Hi Trap Q HP Columns (GE Healthcare Bio-Science Corp. Piscataway, N.J.). A realtime TaqMan® PCR assay (ABI PRISM 7700; Applied Biosystems, Foster City, Calif.) with primers designed to amplify the vector-specific bovine growth hormone polyadenylation sequence was used to determine the concentration of virus particles containing genomes. This concentration is expressed as DNase resistant particles (drp) per ml.

Sensory and Motor Functional Assays. All functional tests were performed after animals had acclimated to the test room for 20 min. Thermal sensory nerve function was monitored with a Hot-Plate Analgesia Meter (Columbus Instruments; Columbus, Ohio) at 50° C. A single animal was placed on the hot plate and timed until it showed a nociceptive response; this time was recorded as its latency to respond. For animals that did not respond prior to a 60 s cutoff time, latency was defined as 60 s. We selected 50° C. because pilot experiments showed that the difference in latency to the heat stimulus between STZ-treated and control animals at later stages of the disease process (hypoalgesia stage) was more significant at 50° C. rather than at 55° C., the standard temperature setting for mice (data not shown).

Nerve conduction velocity was recorded using MP150 System with AcqKnowledge software from BIOPAC Systems, Inc. (Goleta, Calif.). Mice were anesthetized with isoflurane and sensory nerve conduction velocity (SNCV) of the caudal nerve determined. Briefly, a stimulating electrode was placed at the base of the tail and a reference electrode placed 5 mm distally. Recording electrodes were placed on the tail 1 and 2 cm distally with respect to the stimulating electrode. The caudal nerve was stimulated with a single square wave pulse, 0.1 ms in duration and 4 volt intensity. The latencies of the potential detected at the two recording sites after nerve stimulation were determined (peak to peak), and the tail SNCV was calculated accordingly (in m/s). The entire procedure required <15 min.

Rearing activity, a measure of motor function, was determined as previously reported. Briefly, individual mice were placed in Plexiglas cages surrounded by photobeams to capture activity (Opto-Micro Animal Activity System; Columbus Instruments, Columbus, Ohio). Rearing (breaks of beams placed high) was recorded for 15 min and quantified in 5-min bins.

Histology. Mice were euthanized at the end of studies, 130 days post STZ. The spine at the lumbar (L4-6) and sacral (S1-2) levels, and TA muscle were fixed in 10% Neutral Buffer Fix solution (NBF), embedded in paraffin (the spine was decalcified before embedding), and sectioned (3 and 5 μm for the spine, and 5 μm for TA) on a microtome using standard techniques. Slides were stained with standard procedures, Hematoxylin and Eosin (H/E), or Luxol Fast Blue (LFB) and Hematoxylin (LFB/H) for the spine, and H/E for TA. LFB was used to stain myelin.

Sensory Nerve Functional assays. von Frey analyses were performed using an electronic von Frey apparatus (Model 2390; IITC Inc., Woodland Hills, Calif.) and standard procedures. Animals were placed in elevated, clear-plastic, wire mesh-bottomed cages and their paws accessed from the underside of the mesh. All animals were tested after a 20 minute acclimation period. The von Frey filament attached to a sensor was applied perpendicularly to the plantar surface of the hind paw with increasing force for up to 6 seconds. The force provoking an abrupt paw withdrawal was recorded. Each animal was tested three times with at least a 10 min interval between tests. Unlike STZ rats, it was very difficult to detect hypoalgesia in mice; the von Frey test was therefore only used to detect hyperalgesia.

A cold water test was based on a previous report with modification. The system contained two water baths, thermocirculator (Model GD120; Harvard Apparatus, Holliston, Mass.), and a cage with wire bottom that allowed the animal's feet to touch the water when the cage was placed in the water bath. First, a single animal was placed in the cage for 15 min to allow it to acclimate; the cage was then transferred to a water bath containing water at room temperature and the animal acclimated for 5 to 15 min. Finally, the cage was moved to another water bath containing cold water (10° C.). Nociceptive responses (maintaining a rearing position for >10 s) were observed and their latency recorded. The cutoff time (60 s) was recorded if the animal did not respond prior to the cutoff time. Six weeks after STZ treatment, the treated animals began to show rearing activity even in room temperature water. Therefore this test was used only within 6 weeks post STZ to detect hyperalgesia and initial hypoalgesia.

### SUMMARY

Elevated circulating levels of isoform IGF-1 were generated by delivering a plasmid or AAV vector encoding a mouse derived IGF-1 Eb isoform (SEQ ID NO: 4) to the liver by systemic injection. Treatment-induced increases in body weight confirmed that the IGF-1 produced from the plasmid and AAV vectors was bioactive. In the prevention study, treating mice with IGF-1 plasmid at the early hyperalgesia stage largely prevented the subsequent STZ-induced hyposensitivity in this mouse model. Sensory nerve function was indistinguishable from that observed in normal control mice. IGF-1 treatment also increased muscle mass and improved overall mobility as measured by rearing activity, indicating a treatment-related improvement in motor function. Histological analyses showed that the treatment attenuated vacuolization of Schwann cells in the sensory nerve fibers. In the treatment study, AAV-IGF-1 vector was administrated to STZ-treated mice that had advanced to the hyposensitivity stage. Hyposensitivity was reversed after vector administration, with concomitant improvements in rearing activity, muscle mass and overall animal health.

### Early Stage Intervention at the Hyperalgesia Stage:

Early treatment with IGF-1 does not correct hyperglycemia but prevents later hypoalgesia and improves mobility. Nine days after STZ treatment, STZ-induced mice were injected using a high volume plasmid injection technique that mediates robust gene transfer to the liver. Each mouse was injected with 10 ug of plasmid DNA comprising the mouse-derived IGF-1Eb cDNA operably linked to two copies of a human prothrombin enhancer and a human serum albumin promotor.

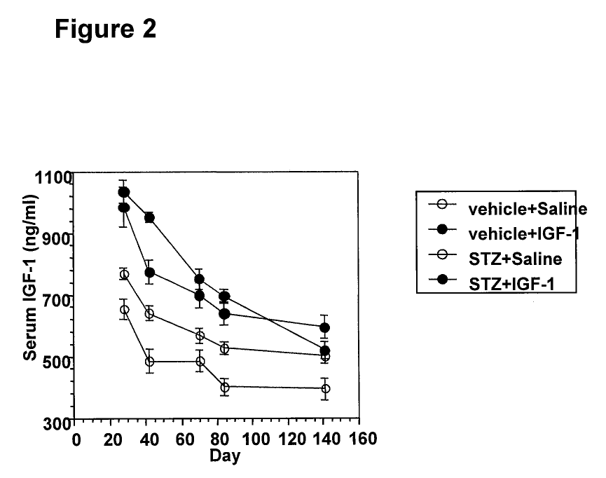

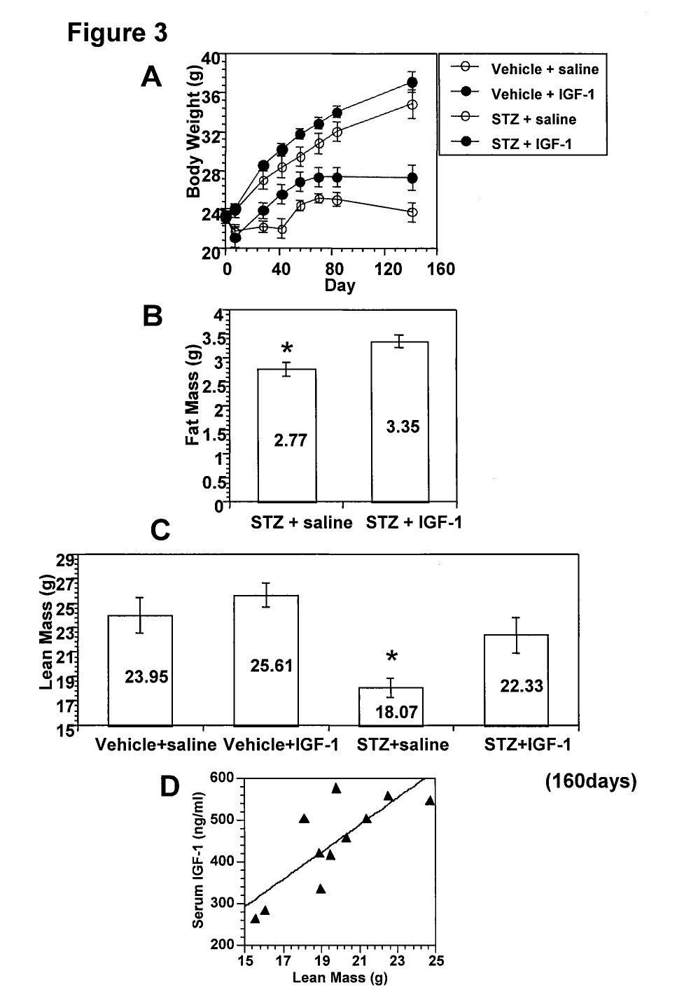

Serum IGF-1 levels in diabetic mice were decreased as compared to non-diabetic control mice (FIG. 2). Diabetic mice that were injected with the plasmid had significantly elevated serum levels of IGF-1 (FIG. 2). Bioactivity of IGF-1 in vivo was confirmed by increases in mouse body weight (FIG. 3A), which was due to a slight increase in fat mass (FIG. 3B) and a larger increase in lean body mass (FIG. 3C). Increases in lean body mass correlated with serum IGF-1 levels (FIG. 3D; p<0.01).

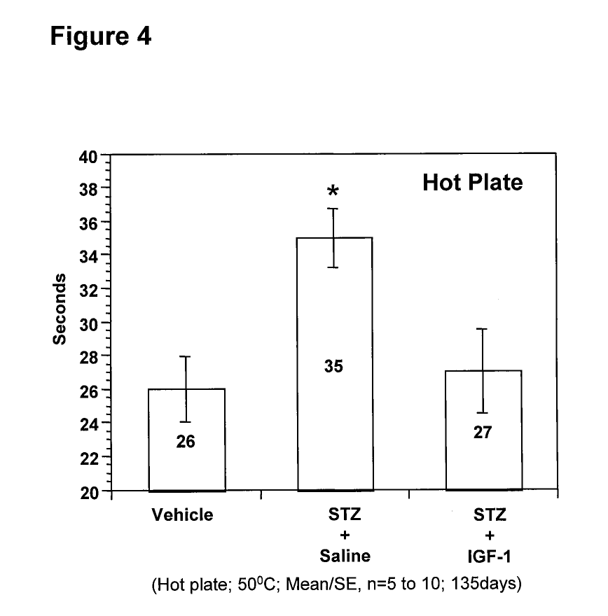

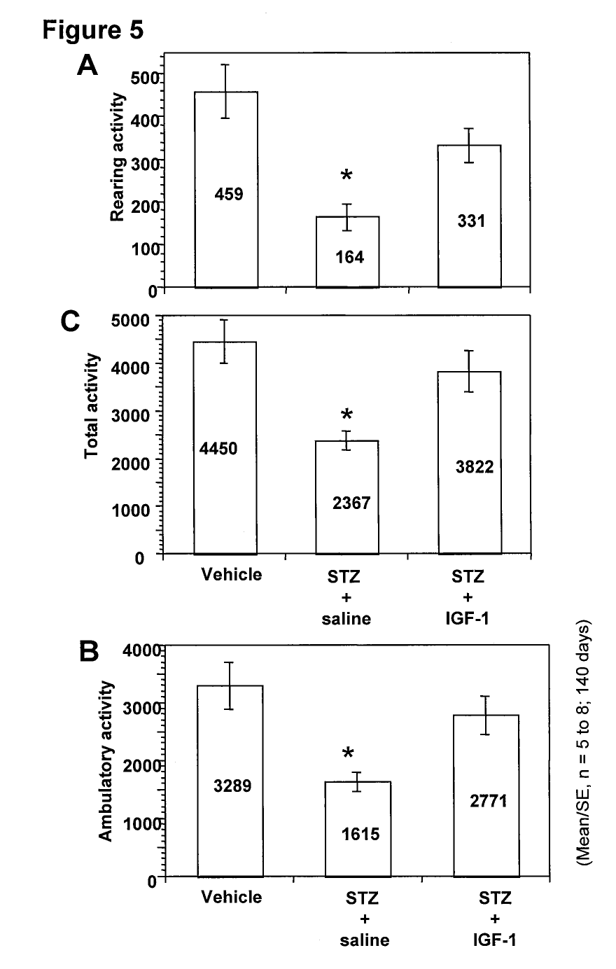

Sensory function in the late stage of DPN was measured using the hot plate assay. Sensory function of IGF-1 treated mice was indistinguishable from that of normal, non-diabetic mice (FIG. 4). Activity overall was significantly decreased in the STZ-treated diabetic mice (FIG. 5C), especially rearing activity (FIG. 5A), which may be related to motor function disorder in the model. IGF-1(Eb) treatment in STZ-diabetic mice normalized total mouse activity (FIG. 5C), ambulatory activity (FIG. 5B), and especially rearing activity (FIG. 5A). Taken together, these results suggest that the early IGF-1 treatment prevented the development of long-term hyposensitivityhypoalgesia and muscle motor function deficits in the diabetic mice.

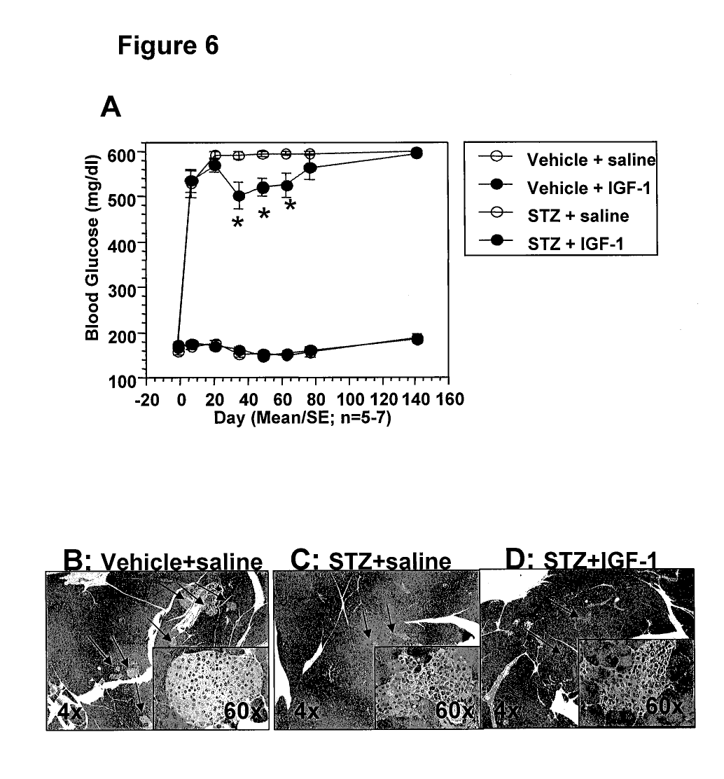

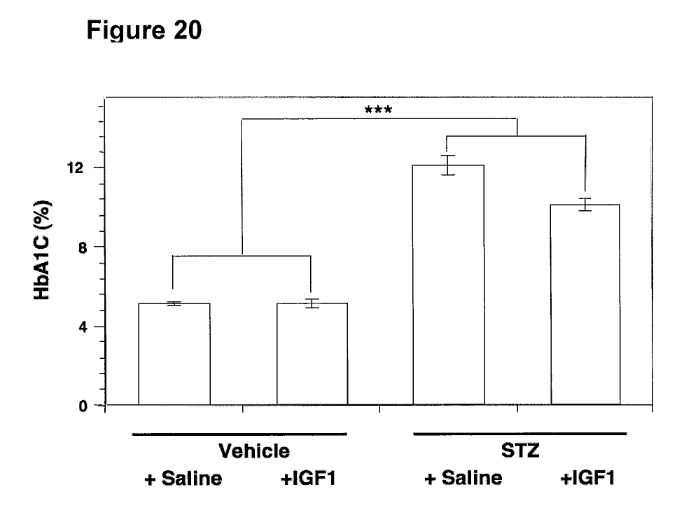

As shown in FIG. 6A, although IGF-1(Eb) treatment had a transient effect on blood glucose levels, it did not correct hyperglycemia. Glucose levels in mice were maintained above 300 mg/dl, which is hyperglycemic. This fact was confirmed by hemoglobin A1C (HbA1c) levels. FIG. 20 shows that HbA1c levels in IGF-1 treated diabetic mice (10±0.5%) remained extremely high, and were not significantly different from those of diabetic mice treated with saline (12±0.3%). IGF-1Eb treatment also did not increase the number or size of pancreatic islets in diabetic mice suggesting that the effects of IGF-1 on DPN are independent of its effects on hyperglycemia (FIG. 6B-6D; arrows point to pancreatic islets).

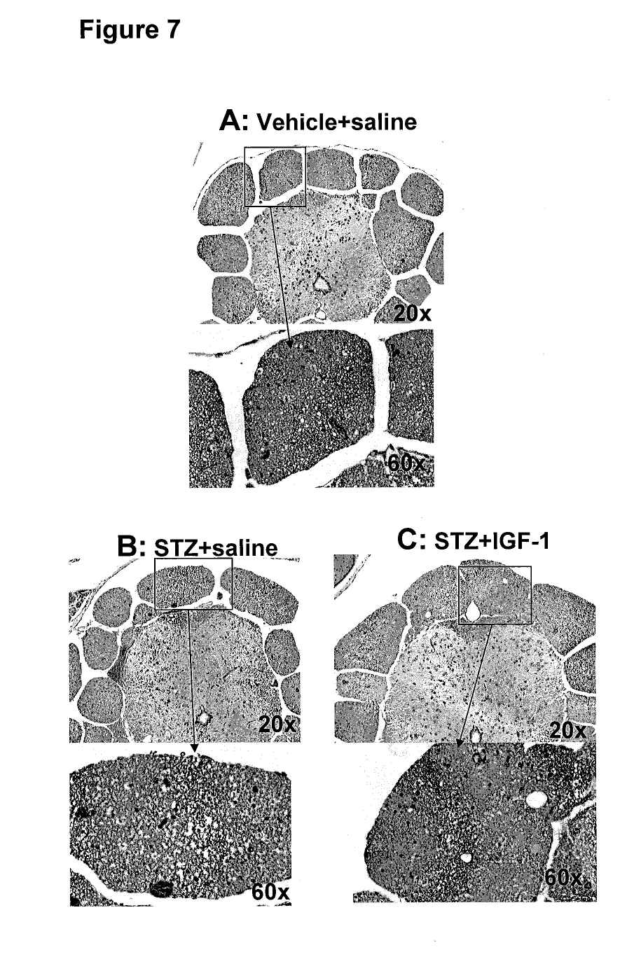

These functional effects of early IGF-1 treatment were corroborated by histologic findings. Vacuolated Schwann cells in peripheral nerves occur in DPN patients and animal models. This was observed in the STZ-treated, diabetic mice in the sensory nerve fibers near the dorsal side of the spinal cord at the sacral level. This pathologic change was attenuated in diabetic mice treated with IGF-1Eb (FIG. 7). For example, FIG. 7 shows that compared to their non-diabetic counterparts (FIG. 7A) vacuolated Schwann cells could be seen in the sensory nerve fibers of untreated diabetic mice (FIG. 7B) upon sacrifice at day 130 post STZ. This vacuolization was apparent in dorsal spinal nerves at both the lumbar and sacral levels. In contrast, Schwann cell vacuolization was significantly attenuated in diabetic mice that had been treated with IGF-1 early in the disease process (FIG. 7C). Since Schwann cell integrity is critical for peripheral nerve myelination and function, these histologic findings are consistent with the STZ-mediated loss of sensory function noted in the diabetic animals and the ability of IGF-1 to preserve this function.

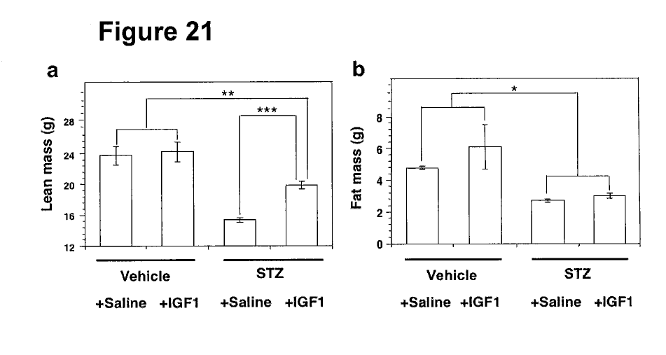

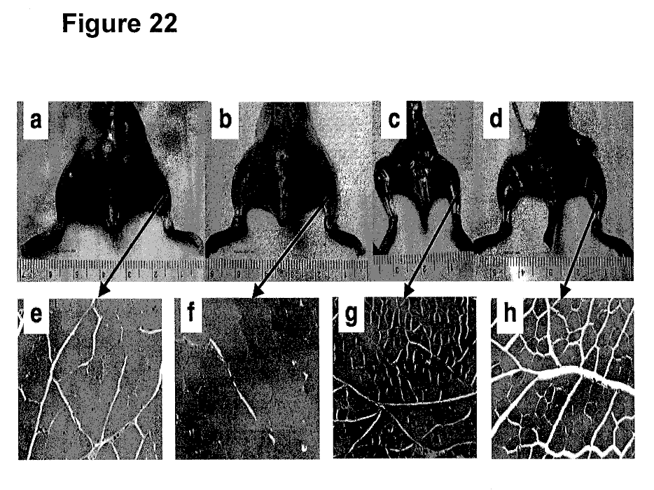

The effects of long term hyperglycemia in the diabetic mice on activity could also be correlated with effects on skeletal muscle mass. Thus, FIG. 21 shows that compared to vehicle-treated controls, diabetic animals exhibited significant long term deficits in both lean and fat mass. Early treatment with IGF-1 resulted in significant increases in the lean but not the fat mass of diabetic animals, consistent with the known anabolic effects of IGF-1. Consistent with these body mass results, FIG. 22 A-D shows that IGF-1 treatment prevented the STZ-induced loss of skeletal muscle. FIG. 22 E-H demonstrates histologically using the tibialis anterior (TA) muscle that the skeletal muscle of diabetic animals was atrophied, ie., individual fiber cross sectional area was reduced, and that this atrophy was significantly attenuated by the early IGF-1 treatment.

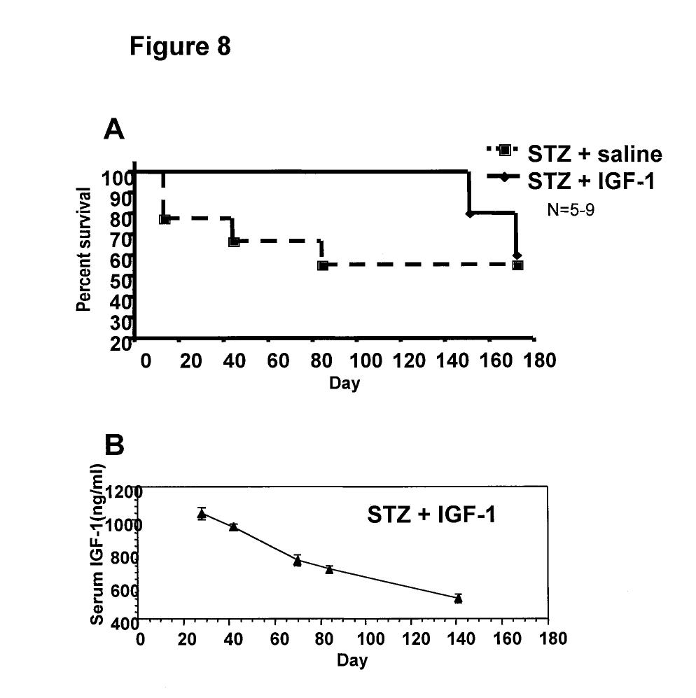

Finally, IGF-1Eb treated, diabetic mice survived significantly longer than untreated mice at early time points (FIG. 8).

### Late Stage Intervention at the Hyposensitivity Stage:

To follow-up these results of treating diabetic mice early in the disease process with IGF-1, we asked whether systemic IGF-1 treatment would prove to be beneficial if delivered late in the disease process, ie., after the diabetic animals had developed demonstrable hypoalgesia. Sixty days after STZ treatment when the mice have developed hyposensitivity, diabetic mice were injected AAV-IGF-1 at one of three doses: 3e9 dnase-resistant particles (drp)/mouse, 3e10 drp/mouse, and 3e 11 drp/mouse to supply sustained blood levels of IGF-1. The AAV vector comprises the mouse IGF-1(Eb) cDNA operably linked to two copies of a human prothrombin enhancer and a human serum albumin promoter.

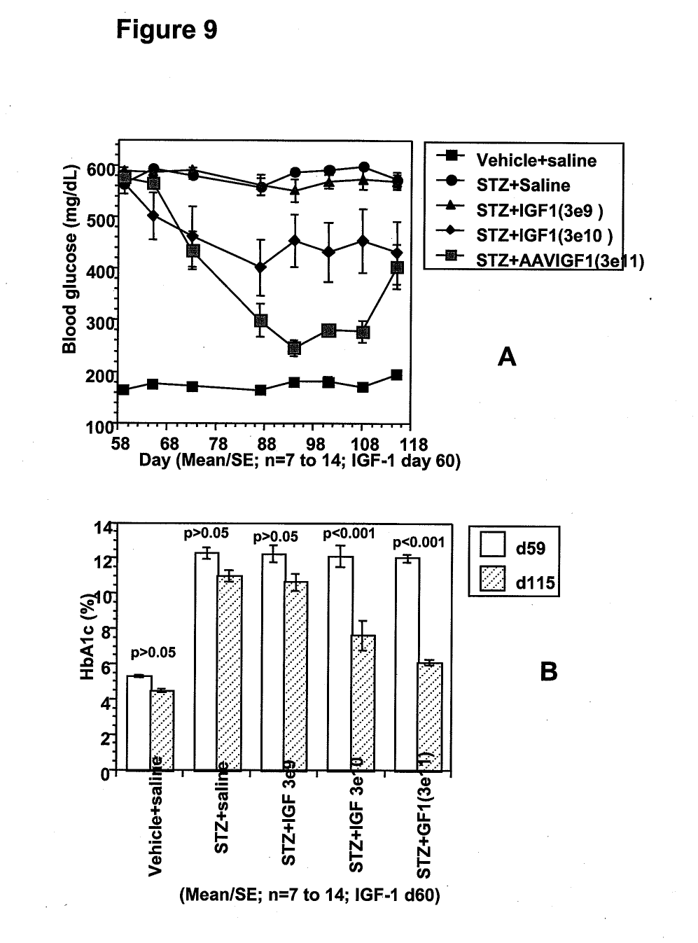

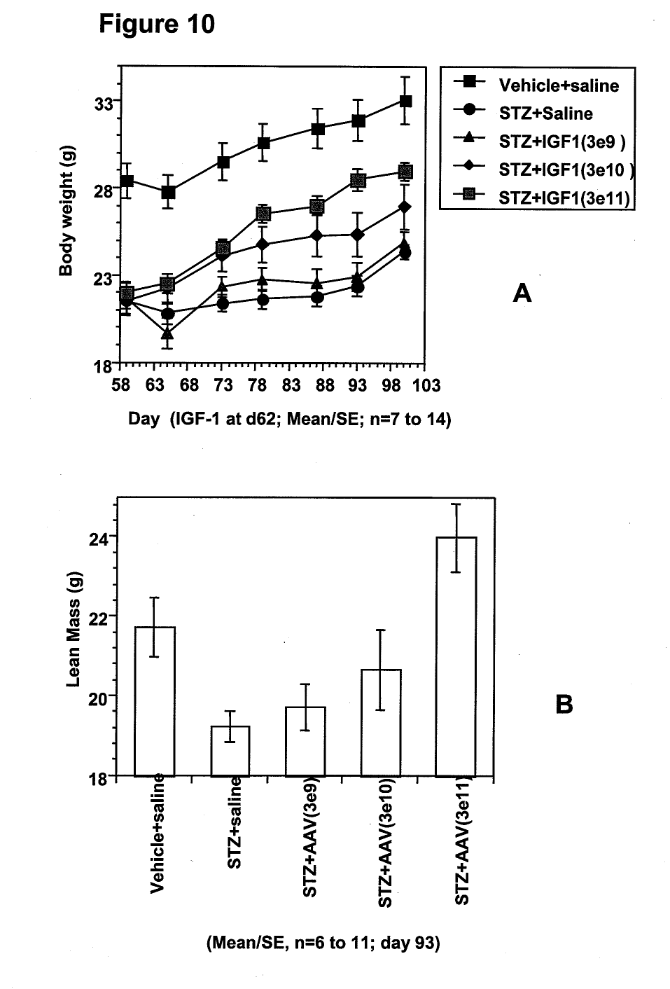

Compared to control mice, STZ treatment led to consistently lower IGF-1 blood levels over time. Treating these diabetic mice with increasing doses of AAV-IGF-1 led to a dose dependent increase in serum IGF-1, viz. a 3E9 dip dose resulted in essentially normal levels of IGF-1, while a 3E11 dose led to IGF-1 levels ˜2 fold normal. At the higher doses, AAV-IGF-1 injection corrected hyperglycemia but did not correct hyperglycemia at the 3e9 vg/mouse dose (FIG. 9). Similarly, at the higher doses, AAV-IGF-1 injection increased body weight and muscle mass of diabetic mice but did not mediate similar effects in these mice at the 3e9 vg/mouse dose (FIG. 10). At this late treatment time point (day 60), the STZ mice weighed significantly less than their control counterparts, and treatment with increasing doses of AAV-IGF-1 led to dose dependent increases in body mass over time.

Also compared to controls, the STZ diabetic mice were severely hyperglycemic, with sustained blood glucose levels of ˜600 mg/dL. The lowest dose of AAV-IGF-1 had essentially no effect on these blood glucose levels, while increasing doses of vector resulted in dose-dependent decreases in blood glucose. These blood glucose results were entirely consistent with parallel HbA1C measurements

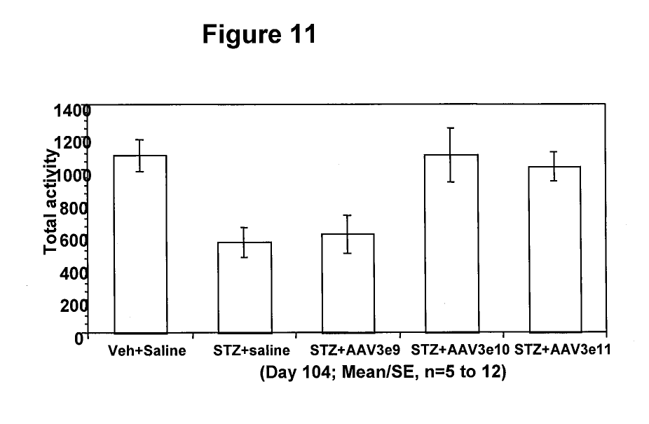

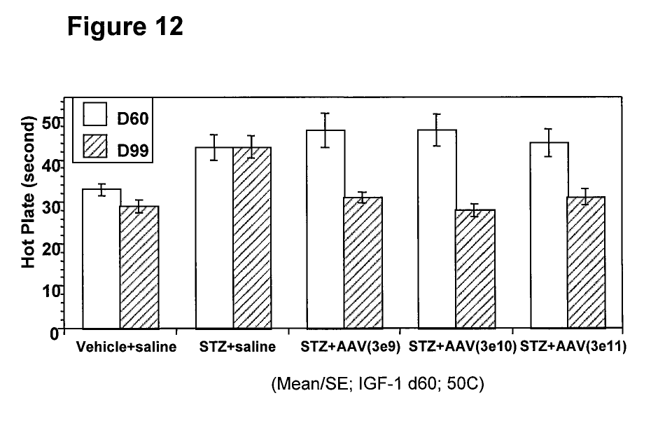

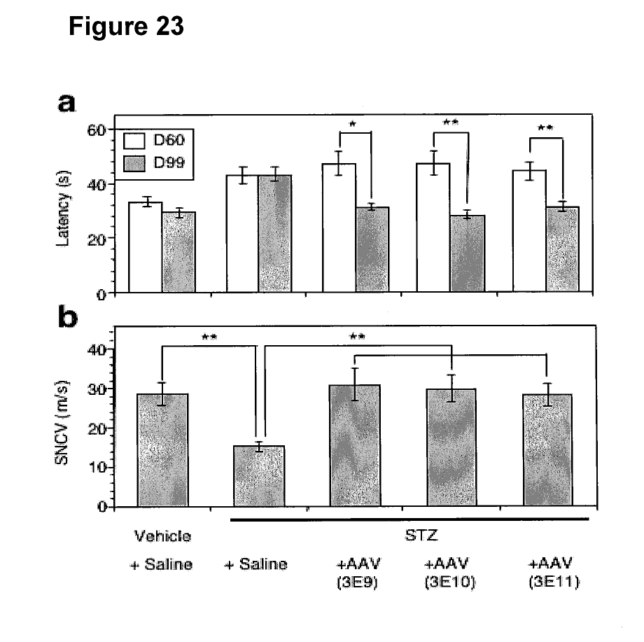

AAV-IGF-1 injection at the higher doses also improved total activity in diabetic mice, but did not improve activity in diabetic mice at the 3e9 vg/mouse dose (FIG. 11). In addition, the efficacy of systemic IGF-1 treatment during hypoalgesia was evaluated using hot plate and sensory nerve conduction velocity (SNCV) assays. Just prior to IGF-1 treatment (at day 60 post STZ), diabetic animals displayed a significantly increased latency time in the hot plate assay compared to controls. At day 99, untreated diabetic animals (STZ+Saline) remained hyposensitive by this measure. In contrast, for all groups treated with IGF-1, latency was restored to normal (FIG. 23A). All three dose levels of AAV-IGF-1 reversed hyposensitivity as measured by the hot plate test in diabetic mice (FIG. 12). Importantly, this was the case even at the lowest dose of AAV8-IGF-1, which normalized serum IGF-1 levels but had no effect on blood glucose or body mass. Apparently, this sensory nerve response was maximal at normalized IGF-1 serum levels, because higher doses of vector had no further effect on latency (FIG. 23A). Therefore, even at the late stage of intervention, IGF-1 treatment reverses hyposensitivity in diabetic mice. This effect appears to be independent from its effects on hyperglycemia.

These observations using the hot plate assay were consistent with results obtained using SNCV measurements. Thus, FIG. 23B demonstrates that at day 115 post STZ (55 days post IGF-1 treatment), diabetic mice had a significantly slowed conduction velocity compared to control mice, (15.2±4.74.1 vs 28.4±10.53.2 m/s, respectively). As with the hot plate assay, normalizing serum IGF-1 levels was sufficient to correct conduction velocity.

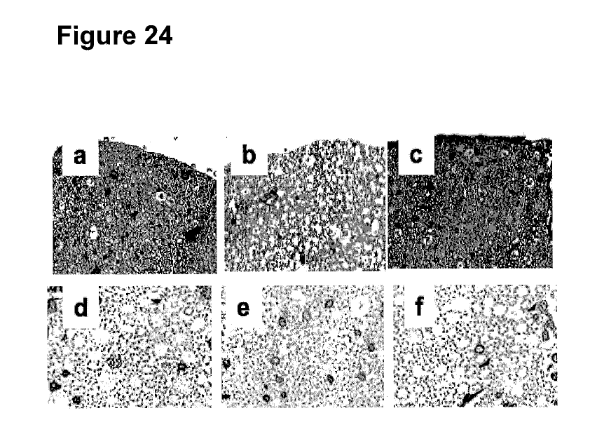

Findings from a histologic examination of dorsal spinal nerves at the lumbar and sacral levels were consistent with these assays of sensory nerve function. Thus, FIG. 24 shows that compared to the appearance of nerve fibers in normal animals (FIG. 24 A, D), fibers from diabetic mice showed vacuolated Schwann cells (FIG. 24B) and disrupted myelin sheaths (FIG. 24E). In contrast, fibers from diabetic mice that had been treated with IGF-1 after developing hypoalgesia demonstrated essentially normal Schwann cell (FIG. 24C) and myelin (FIG. 24F) morphology. These beneficial effects were dose-dependent, with 7/7 animals demonstrating normal morphology at a dose of 3E11 drp, 6/6 at 3E10, and 3/5 at 3E9.

Taken together, these data demonstrate that when administered after the onset of hypoalgesia, the lowest dose of AAV-IGF-1, which normalized serum IGF-1 levels, had essentially no effect on either weight gain or blood glucose. Higher doses of IGF-1 resulted in proportionately larger increases in body mass and more significant decreases in blood glucose compared to saline-treated controls. These results indicate that treating severely diabetic mice with systemic IGF-1 can actually reverse existing hypoalgesia, due at least in part to its beneficial effects on Schwann cells. Importantly, these effects on sensory neurons function were maximal when serum IGF-1 levels were simply normalized, ie. an increase of ˜100 ng/ml over IGF-1 levels in diabetic mice. Increasing serum IGF-1 above the normal level could further correct morphological changes of peripheral sensory nerves.

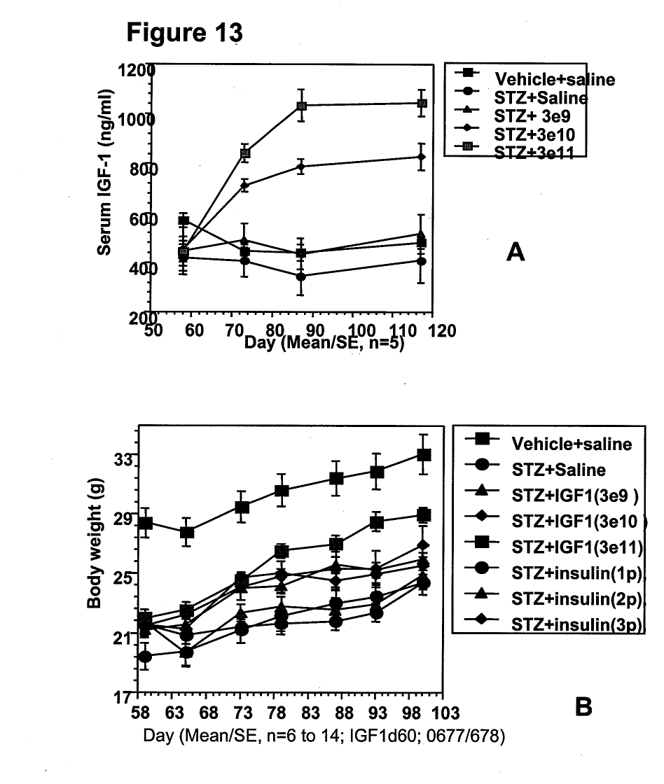

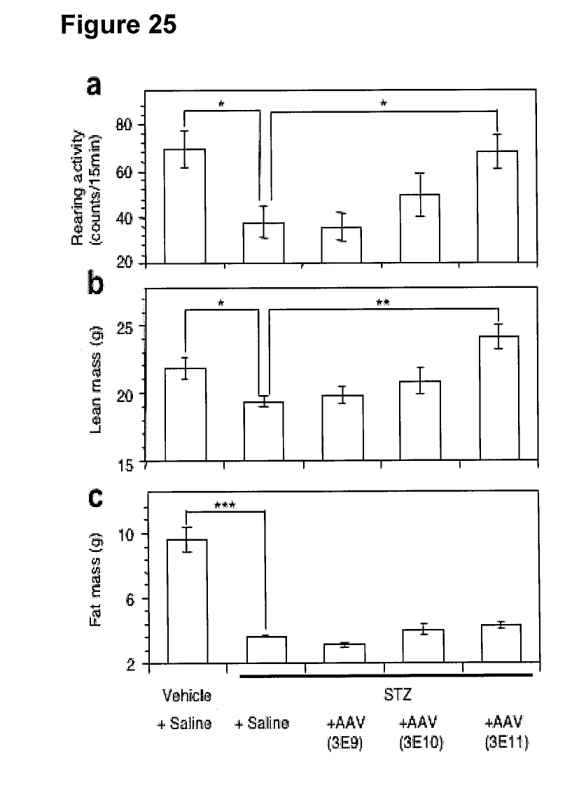

Impaired motor function responds only to supranormal IGF-1 levels. FIG. 25A shows that as a measure of motor function, rearing activity, was significantly decreased in diabetic mice at day 100 compared to controls. In contrast to the correction of sensory function at the lowest dose of AAV-IGF-1 (above), rearing activity responded only to higher doses of AAV-IGF-1, ie. at doses where serum IGF-1 was sustained at supranormal levels (FIG. 13). At the highest serum IGF-1 levels rearing activity was restored to normal.

These effects of IGF-1 on motor function were mirrored by its effects on lean mass. Thus, FIGS. 25B and 25C show that the diabetic condition resulted in significant decreases in both lean (FIG. 25B) and fat (FIG. 25C) mass. In parallel to the dose dependent effects of IGF-1 on rearing activity, FIG. 25B shows that the low dose of AAV-IGF-1 had only a minimal effect on lean mass, while the highest dose restored lean mass to at least normal levels. Effects of IGF-1 were restricted to effects on lean mass, as FIG. 25C shows that fat mass was not affected significantly even at the highest dose of AAV-IGF-1.

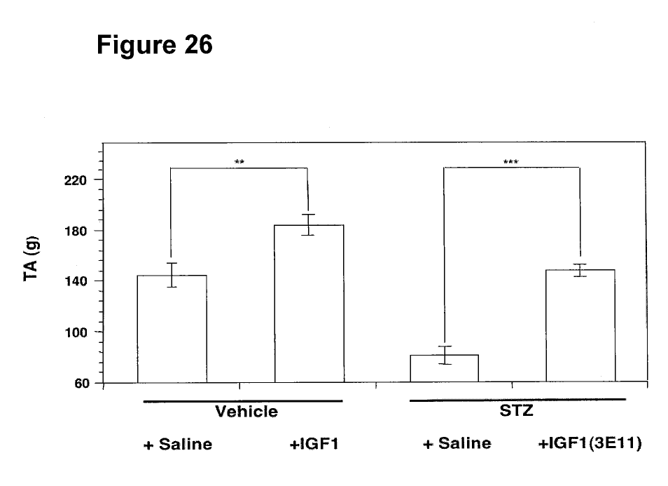

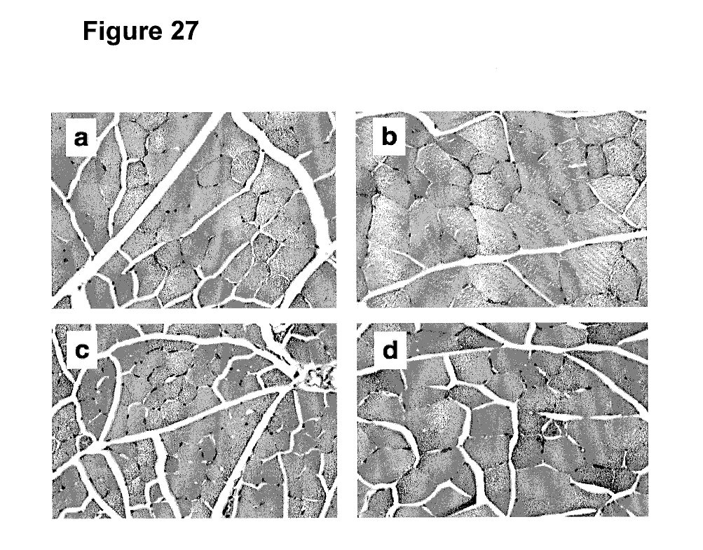

To confirm and extend these apparent effects of IGF-1 on skeletal muscle, the tibialis anterior (TA) muscle was examined in more detail. FIG. 26 shows that at the highest dose of IGF-1, the TA mass of diabetic animals was restored to normal, consistent with the effects of IGF-1 in these animals as measured by lean mass. FIG. 27 shows that this IGF-1-mediated increase in TA mass appeared to be the due to preventing the atrophy seen in muscle fibers of untreated diabetic mice, as documented by a histologic examination of fiber size.

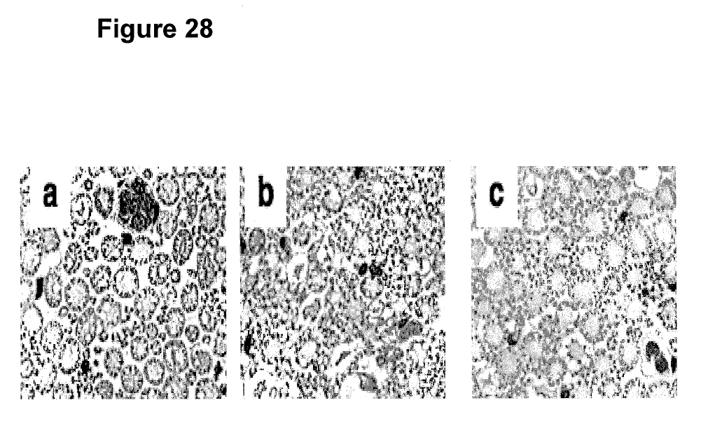

Finally, FIGS. 28A and 28B shows histologically that ventral motor nerve fibers at the lumbar and sacral levels of the spinal cord in the diabetic mice had undergone significant demyelination (day 130 post STZ). FIG. 28C shows that this demyelination was could be significantly attenuated by IGF-1 treatment during the hypoalgesia stage of the disease. This beneficial effect of IGF-1 on demyelination was dose-dependent, with 7/7 showing clear improvement at the highest dose, 5/6 at 3E10, and 3/5 at 3E9 drp/mouse.

Taken together, these data support the notion that restoring serum IGF-1 levels to normal, even after a state of hypoalgesia has been established, can reverse sensory but not motor nerve function deficits. Supranormal serum levels of IGF-1 prevent skeletal muscle loss and further improve motor nerve structure, resulting in a preservation of and motor function.

### Late Stage Intervention at the Hyposensitivity Stage to Evaluate Renal Effects:

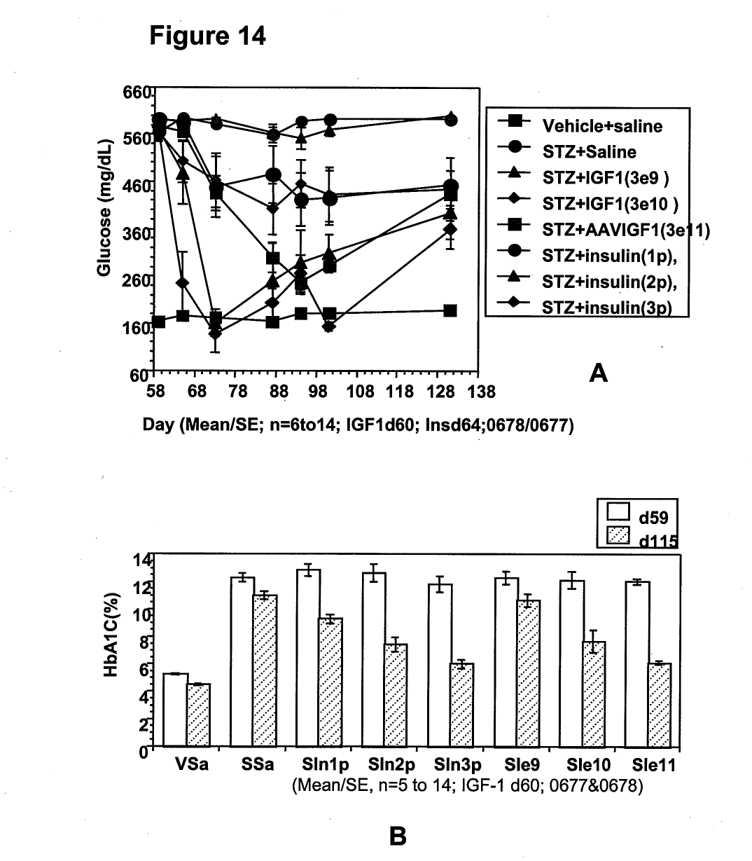

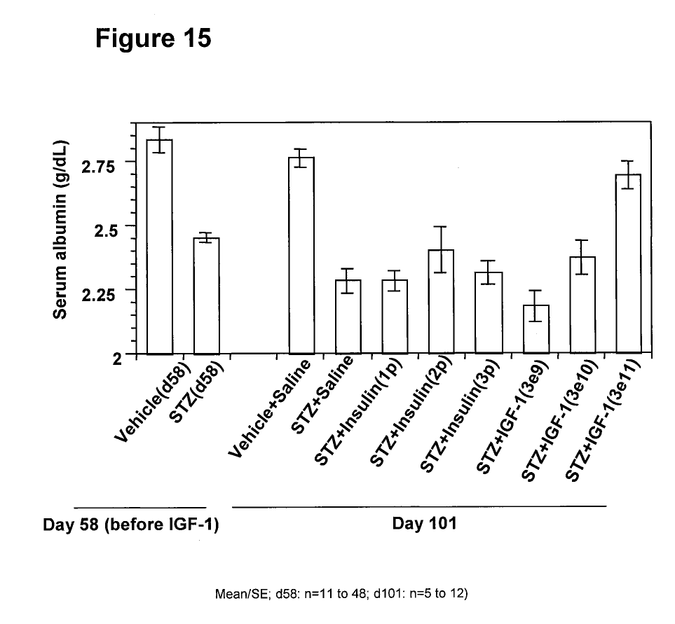

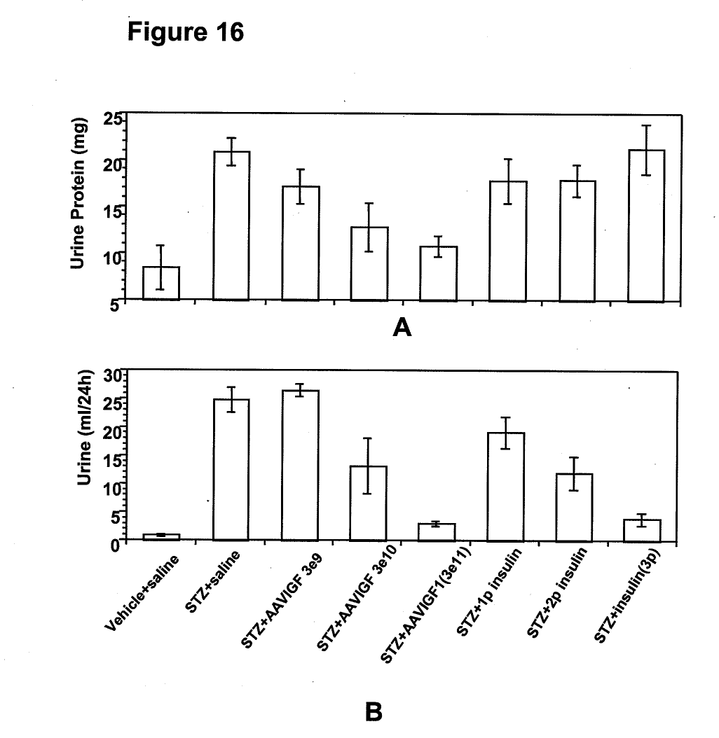

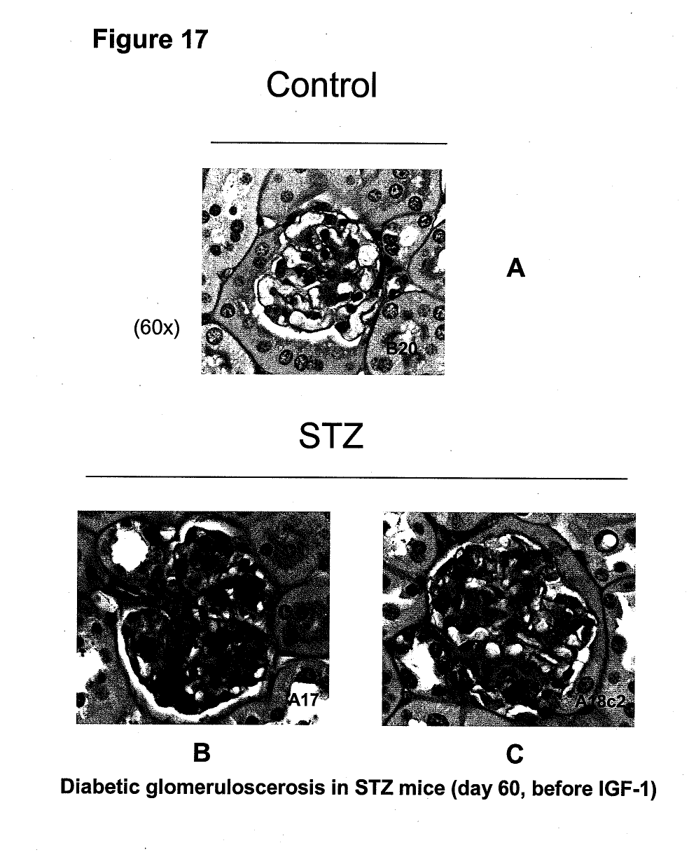

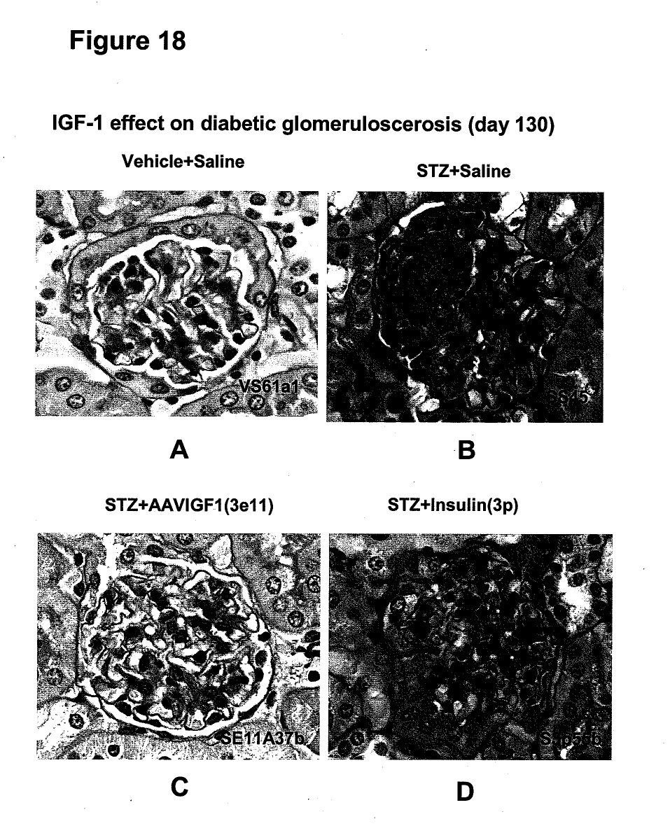

Sixty days after STZ treatment, diabetic mice were injected AAV8-IGF-1 at one of three doses: 3e9 dnase-resistant particles (drp)/mouse, 3e10 drp/mouse, and 3e11 drp/mouse. The AAV vector comprises the mouse IGF-1(Eb) cDNA operably linked to two copies of a human prothrombin enhancer and a human serum albumin promoter. Insulin pellet treatment was included as a control. Serum IGF-1 levels, blood glucose, body weight, HbA1c, renal function and renal histology were investigated. Serum IGF-1 levels in diabetic mice were decreased as compared to non-diabetic control mice. Diabetic mice that were injected with the higher doses of AAV-IGF-1 had significantly elevated serum levels of IGF-1. Serum IGF-1 levels and its effect on body weight are shown in FIG. 13. Increases in body weight mediated by IGF-1 were more significant than those mediated by insulin treatment. Comparing the high dose of AAV-IGF-1 with the high dose of insulin, both significantly decreased blood glucose and corrected HbA1c although the insulin treatment had more effect on blood glucose (FIG. 14). In measuring renal function, IGF-1 treatment brought serum albumin close to control levels an effect not observed in mice receiving insulin only (FIG. 15). Although both IGF-1 and insulin treatments showed dose-dependent reduction for urine volume, urine protein was significantly decreased only in IGF-1 treated diabetic mice (FIG. 16). In evaluating renal histology, diabetic glomerulosclerosis was reversed with IGF-1 treatment, but not with insulin (compare FIG. 17 demonstrating renal histology pre-IGF-1 treatment to FIG. 18 demonstrating renal histology post-IGF-1 treatment). Our results demonstrate that increase systemic IGF-1 modulates renal function and diabetic glomerulosclerosis in the STZ mouse model of DN.

We claim:

1. A method to treat a subject with diabetic peripheral neuropathy in the hyposensitivity stage comprising administering an effective amount of IGF-1Eb to said subject.

2. A method to treat a subject with diabetic peripheral neuropathy in the hyposensitivity stage comprising administering an effective amount of human IGF-1Ec to said subject.

3. A method to treat a subject with diabetic nephropathy comprising administering an effective amount of IGF-1Eb to said subject.

4. A method to treat a subject with diabetic nephropathy comprising administering an effective amount of IGF-1Ec to said subject.

5. The method of claim 1 or 3, wherein the IGF-1Eb is administered to said subject by protein infusion.

6. The method of claim 1 or 3, wherein the IGF-1Eb is administered to said subject by delivery of a plasmid vector encoding for said IGF-1Eb.

7. The method of claim 1 or 3, wherein the IGF-1Eb is administered to said subject by delivery of a recombinant AAV vector encoding for said IGF-1Eb.

8. The method of claim 2 or 4, wherein the IGF-1Ec is administered to said subject by protein infusion.

9. The method of claim 2 or 4, wherein the IGF-1Ec is administered to said subject by delivery of a plasmid vector encoding for said IGF-1Ec.

10. The method of claim 2 or 4, wherein the IGF-1Ec is administered to said subject by delivery of a recombinant AAV vector encoding for said IGF-1Ec.

11. A method to treat a subject with diabetic peripheral neuropathy in the hyposensitivity stage comprising administering an effective amount of an IGF-1 protein consisting essentially of SEQ ID NO: 4 to said subject.

12. A method to treat a subject with diabetic nephropathy comprising administering an effective amount of an IGF-1 protein consisting essentially of SEQ ID NO: 4 to said subject.
